(19) United States
(12) Patent Application Publication
(10) Pub. No.: US 2022/0267274 A1
(43) Pub. Date: Aug. 25, 2022

(54) TARGETING PTPN22 IN CANCER THERAPY

(71) Applicant: The Johns Hopkins University, Baltimore, MD (US)

(72) Inventors: Elizabeth Jaffee, Baltimore, MD (US); Won Jin Ho, Ellicott City, MD (US)

(21) Appl. No.: 17/625,976

(22) PCT Filed: Jul. 10, 2020
(86) PCT No.: PCT/US2020/041544

§ 371 Date: Jan. 10, 2022

Related U.S. Application Data
(60) Provisional application No. 62/872,306, filed on Jul. 10, 2019.

(51) Int. Cl. C07D 215/56; A61P 35/00; A61K 31/713; A61K 45/06; A61K 31/47

(52) U.S. Cl. CPC C07D 215/56; A61P 35/00; A61K 31/47; A61K 45/06; A61K 31/713

(57) ABSTRACT

Described are methods of treating solid cancers in a subject. The methods comprise the steps of administering to the subject having the solid cancer or prone of getting the solid cancer an antagonist of PTPN22, or the functional part of PTPN22, and treating the solid cancer. Methods comprising use of other anticancer agents and adjuvants in conjunction with PTPN22 inhibitors are also provided.

### REFERENCE TO RELATED APPLICATIONS

This application claims the benefit of U.S. Provisional Patent Application No. 62/872,306, filed on Jul. 10, 2020, which is hereby incorporated by reference for all purposes as if fully set forth herein.

### STATEMENT OF GOVERNMENTAL INTEREST

This invention was made with government support under grant nos. P50CA062924 and 5T32CA009071-38 awarded by the National Institutes of Health. The government has certain rights in the invention.

### BACKGROUND OF THE INVENTION

Recent progress in cancer immunotherapy has revolutionized the management of several cancer types³. Understanding the importance of T cells in anticancer immunity has provided the foundation for many therapeutic options including checkpoint inhibition and adoptive T cell approaches, e.g. chimeric antigen receptor T (CAR T) cell therapies⁴⁻⁶. However, the majority of patients either have a very limited or altogether lack response to current immunotherapies, warranting the ongoing search for additional immunotherapy strategies.

The fundamental process within T cells that links antigen recognition to cellular output is the T cell receptor (TCR) signaling cascade. Protein tyrosine phosphatase non-receptor type 22 (PTPN22), a member of the PTP superfamily that is largely specific to immune cells, is a physiologic regulator of TCR signaling^{1,2}. By dephosphorylating the activating tyrosine residues of Lck (Y394) and Zap70 (Y493), which are proximal kinases of the TCR signaling cascade, PTPN22 is able to subdue the activity of TCR signaling. In fact, numerous epidemiologic studies have established the association between a germline variant of PTPN22, C1858T (R620W; rs2476601), and autoimmune diseases⁸⁻¹⁰. Accordingly, mouse studies have demonstrated that PTPN22 R619W (homologous to human R620W) is consistent with the loss-of-function phenotype seen in PTPN22 KO mice^{11,12}. Given the well-studied homeostatic role of PTPN22 in autoimmune pathophysiology, we hypothesized that PTPN22 is a systemic target for augmenting antitumor immune responses.

However, neither the expression of PTPN22 in the tumor microenvironment (TME) nor its potential role as a suppressor of systemic antitumor immunity has been well characterized. Thus, the inventors describe for the first time a successful therapeutic strategy of targeting PTPN22 to enhance antitumor immune responses.

### SUMMARY OF THE INVENTION

Herein the inventors now have validated PTPN22 as a target for cancer immunotherapy. The inventors also found that individuals with the PTPN22 rs2476601 variant (C1858T; R620W) not only are at increased risk for autoimmune disease, but also have a reduced incidence of cancers. Using in vivo models, the inventors demonstrate that genetic ablation of PTPN22 leads to augmented antitumor immune responses and response to PD1 inhibition. The inventors also developed a novel small molecule inhibitor of PTPN22 and show that pharmacologic inhibition of PTPN22 recapitulates this phenotype. Furthermore, patients with rs2476601 variant associate with significantly greater responses to checkpoint immunotherapy. Mechanistically, targeting of PTPN22 synergizes with immune checkpoint inhibitor therapy as shown by immune and phosphoproteomic profiling. The described results provide a new molecular target that may induce and enhance responses to current immunotherapies. Furthermore, when combined with anti-PD1 checkpoint inhibitor, which is a standard immunotherapy agent used for the treatment of multiple types of cancers, the PTPN22-knockout mice have significantly augmented suppression of tumor growth compared to that of PTPN22-wildtype mice. This suggests that PTPN22 is a target for immunotherapeutic treatment of cancers and that combining anti-PTPN22 strategy with anti-PD1 therapy should be more effective than using either agents alone.

Importantly, the inventors also show for the first time that the proof-of-concept that another small molecule inhibitor of PTPN22, LTV1 (commercially available), can be utilized to augment antitumor immune responses. The inventors were surprised to discover that systemically targeting of PTPN22 augmented antitumor responses and enhanced the effects of checkpoint immunotherapy, and anticipate similar enhancement with co-stimulatory agonist immunotherapy, antitumor vaccination, and T cell transfer therapy. To assist with measuring changes in the TCR signaling, the inventors developed a phospho-flow cytometry assay to measure the changes in the phosphorylation states of two PTPN22 enzymatic substrate kinases, Lck Y394 and Zap70 Y493, in T cells.

Therefore, in accordance with a first embodiment, the present invention provides a method of treating cancer in a subject comprising administering to the subject an effective amount of an inhibitor of PTPN22.

In accordance with a second embodiment, the present invention provides a method of treating cancer in a subject comprising administering to the subject an effective amount of an inhibitor of PTPN22 comprising the compound LTV1, or a salt, solvate or stereoisomer thereof.

In accordance with a third embodiment, the present invention provides a method of treating cancer in a subject comprising administering to the subject an effective amount of an inhibitor of PTPN22 comprising compounds of Formulas (I), (II), (III), and (IV).

In accordance with a fourth embodiment, the present invention provides a method of treating cancer in a subject comprising administering to the subject an effective amount of an inhibitor of PTPN22 comprising the compound L1, or a salt, solvate or stereoisomer thereof.

In accordance with a fifth embodiment, the present invention provides a method of treating cancer in a subject comprising administering to the subject an effective amount of an inhibitor of PTPN22 and an effective amount of at least one additional anti-cancer agent.

In accordance with a sixth embodiment, the present invention provides a method of treating cancer in a subject comprising administering to the subject an effective amount of an inhibitor of PTPN22 and an effective amount of at least one checkpoint inhibitor.

### BRIEF DESCRIPTION OF THE DRAWINGS

FIGS. 1A-1C. PTPN22 is associated with a negative regulatory role in the immune response against cancer. 1A, Volcano plot showing the results from the analysis using phenome-wide association studies (PheWAS) from the Vanderbilt BioVU database. Each dot represents association between PTPN22 rs2476601 and a disease diagnosis. Horizontal dashed line marks FDR-adjusted p-value of 0.05. 1B, Correlations between PTPN22 expression and immune cell types deconvolved by CIBERSORT across 11 cancer types from the Cancer Genome Atlas (TCGA) are shown as a heatmap. 1C, Correlation between PTPN22 and immune regulatory markers across 11 cancer types from TCGA are shown as a heatmap. Abbreviations: bladder cancer, BLCA; breast cancer, BRCA; colon adenocarcinoma, COAD; glioblastoma multiforme, GBM; head and neck squamous cell cancer, HNSC; hepatocellular carcinoma, LIHC; lung adenocarcinoma, LUAD; lung squamous cell carcinoma, LUSC; pancreatic ductal adenocarcinoma, PAAD; prostate adenocarcinoma, PRAD; skin melanoma, SKCM.

FIG. 2A-2D PTPN22 correlates with negative immune regulation in cancers. 2A, Volcano plot of the results from the analysis using phenome-wide association studies (PheWAS) from the Vanderbilt BioVU database focused on de-identified patients of African ancestry. 2B, Heatmap of correlations between Ptpn22 expression and several cell type signatures based on a gene set based algorithm, xCell, across 11 cancer types in the Cancer Genome Atlas (TCGA). 2C, Kaplan-Meier curves represent univariate survival analyses for six genes, Pdcd1, Ctla4, Lag3, Cd4, Cd8a, and Ptpn22, stratifying high-expressor cases (top 20%, blue) vs. the remaining (black) using the melanoma TCGA database. *p<0.05, ***p<0.005, log-rank tests. 2D, Correlation between the Ptpn22 expression and the total number of mutations are shown in aggregate for 11 cancer types from TCGA. Blue line represents the overall correlation strength (0.142). Abbreviations: breast cancer, BRCA; head and neck squamous cell cancer, HNSC; hepatocellular carcinoma, LIHC; lung adenocarcinoma, LUAD; lung squamous cell carcinoma, LUSC; pancreatic ductal adenocarcinoma, PAAD; prostate adenocarcinoma, PRAD; skin melanoma, SKCM.

FIGS. 3A-3H: PTPN22 KO confers protection against MC38 tumor growth in association with enhanced immune infiltration. 3A, Schematic of the mouse tumor model used. 2.5×10⁵ cells were injected subcutaneously in the right hind limb for tumor growth measurements 2-3 times a week through 21 days after injection. 3B, MC38 tumor growth was compared between WT (blue circles) and PTPN22 KO (red squares) using mixed-effect modeling (***p<0.005). Data are mean±s.e.m. (n=10-11 per arm). Pictures on the adjacent panel shows gross morphology of the two representative tumors from each arm. 3C, Weights of the MC38 tumors on the day of the harvest (day 21) from WT and PTPN22 KO mice, mean±s.e.m. (n=15-16). 3D-E, Immunohistochemistry analysis of the tumors compares CD4+ and CD8+ cells (3D) and Foxp3+ cells (3E) infiltrating the tumors (5× magnification). Positive stain was quantified using HALO software, and the results were summarized as dot plots with mean±s.e.m. (n=11-13 per arm). *p<0.05, ***p<0.005, two-tailed unpaired t-tests. 3F-G, Immune subsets (3F) and T cell subsets (3G) are represented as bar graphs as % of live cells with the results of mixed linear modeling annotated (WT vs. KO, mean+s.e.m., n=8, *p<0.05, **p<0.01, ***p<0.005). UMAP plot from CyTOF analysis of the immune profile is shown (3F). 3H, Conventional flow cytometry was performed to validate CyTOF findings showing increased CD8+ T cells and CD4+ T cells within the MC38 tumors in PTPN22 KO mice compared to WT. Data are mean±s.d. (n=4). *p<0.05 and ***p<0.005, one-tailed unpaired t-tests. Abbreviations: checkpoint markers, Ckpt; dendritic cells, DC; granulocytic myeloid-derived suppressor cells/tumor associated neutrophils, G-MDSC/TAN; granzyme, Gz; monocytic myeloid-derived suppressor cells/myeloid cells, M-MDSC/MC; monocytes, MC; natural killer cells, NK; natural killer T cells, NKT; tumor-associated macrophages, TAM; cytotoxic T cells, Tc; helper T cells, Th; regulatory T cells, Treg.

FIGS. 4A-4C: Clustering and functional marker analysis of MC38 tumors from WT vs. PTPN22 KO mice profiled by CyTOF. 4A, The result from the FlowSOM clustering into 30 metaclusters based on 16 canonical subtyping markers and then annotated into 16 final major immune cell types for global immune profiling of the tumors is shown as a normalized metal intensity heatmap. 4B, An in-depth FlowSOM analysis of the T cell population using 21 subtyping and functional markers to yield 30 metaclusters annotated into 9 final T cell subtypes is shown as a normalized metal intensity heatmap. 4C, Functional marker expressions in the MC38 tumors from WT and PTPN22 KO mice are profiled by CyTOF. Box plots represent the mean metal intensities for each of the functional marker expression stratified by the cell types (n=8). Results from mixed linear modeling for differential analysis between the two groups are indicated by FDR-adjusted p values: #<0.2, *<0.1, ***<0.005.

FIGS. 5A-5E: TCR-activation in CD8+ T cells lacking PTPN22 can be further augmented with checkpoint immunotherapy. 5A, Phosphorylation intensities (mean metal intensities) for each of the indicated phospho-site, stratified by the subtype of CD8+ T cells, comparing MC38 tumor-infiltrating CD8+ T cells from WT and PTPN22 KO mice (n=5 mice per arm). Results of linear mixed modeling for differential analyses of the phosphorylation levels are shown as FDR-adjusted p values: *<0.1, ***<0.005. Abbreviations: central memory subtype, CM; effector memory subtype, CM; effector subtype, Eff; “exhausted” subtype (positive expression of checkpoint markers), EX. 5B-C, Dot plots with mean±s.e.m. (n=9-10) for flow cytometry to determine the proportion of tumor infiltrating CD8+ T cells that are Tbethi (5B) and EOMES+(5C). **p<0.01, two-tailed unpaired t-test comparing WT and PTPN22 KO. 5D, Inset: schematic of the experiment testing the effects of a single 200 μg dose of anti-PD1 therapy against MC38 tumors in WT and PTPN22 KO mice. Growth curves (mean±s.e.m., n=5 per arm) are shown for four groups: WT or PTPN22 KO mice treated with isotype antibody (WT ISO or PTPN22 KO ISO) or PD1 antibody (WT aPD1 or PTPN22 aPD1). Isotype treated control groups were euthanized on day 21 from tumor injection. 5E, Response to checkpoint immunotherapy in patients with rs2476601 or WT PTPN22 from the BioVU database. “Combination Therapy” (gray dot) refers to a combination of the indicated immunotherapy with a non-immunotherapeutic drug. Inset: Kaplan-Meier curves comparing PFS.

FIGS. 6A-6F: PTPN22 KO mice resist EG7 tumors significantly better than WT mice and exhibit superior antigen-specific responses. 6A, Schematic of tumor resistance experiment with EG7 tumors: 2.5×10⁵ cells were injected subcutaneously in the right hind limb, and tumor persistence was assessed on day 35. 6B, The frequency of tumors rejected in WT and PTPN22 KO mice are displayed (n=20 per arm). 6C, Both the EG7 tumor weights and volumes measured on day 35 from WT and PTPN22 KO mice without tumor rejection (n=11-15) are shown as mean±s.e.m. Not significant (n.s.), *<0.05, **p<0.01, two-tailed unpaired t-tests. 6D, Results of the tetramer analysis by flow cytometry, mean±s.e.m. SIINFEKL tetramer+CD8+ T cells in the tumor draining lymph nodes from EG7 tumor-bearing mice are compared using one-way ANOVA followed by pairwise Tukey's test (n=9), *p<0.05. 6E, Experimental schematic of the vaccination regimen used to compare antigen-specific responses. 6F, SIINFEKL tetramer+CD8+ T cells from the spleens of vaccinated mice, mean±s.e.m. *p<0.05, ***p<0.005, one-way ANOVA followed by pairwise Tukey's test (n=5).

FIGS. 7A-7E: Treatment with a novel small molecule inhibitor of PTPN22, L-1, phenocopies PTPN22 KO. 7A, Schematic of the L-1 treatment of MC38 and CT26 tumor model. Starting day 3 of injection, L1 is administered intraperitoneally twice daily for five consecutive days per week for two weeks and once daily for five consecutive days per week for one week. Structure of L-1 is illustrated. 7B-C, Tumor growths of MC38 in C57/Bl6J (7B) and CT26 in Balb/cJ (7C) were compared between vehicle (blue circles) and L-1 (red squares) treatment groups using mixed-effect modeling (**p<0.01). Mean±s.e.m. (n=9-10 per arm). 7D, Immunohistochemistry analysis of the MC38 tumors shows CD4+ and CD8+ cells infiltrating the tumors (5× magnification). Positive stain was quantified using HALO software, and the results are shown as mean±s.e.m. (n=6-8 per arm). *p<0.05, two-tailed unpaired t-tests. 7E, To assess potential off-target effects of L-1, starting day 3 of injection, PTPN22 KO mice are given either vehicle (VEH) or L-1 intraperitoneally twice daily for five consecutive days per week for two weeks and once daily for five consecutive days per week for one week. Tumor growth curves for VEH (blue circles) and L-1 (red squares). Mean±s.e.m. (n=7 per arm). Not significant (n.s.), mixed effects modeling.

FIGS. 8A-8I: CyTOF phospho-profiling of CD8 T cells. 8A, Design of the CyTOF phospho-profiling panel, denoting 9 phospho-sites along the TCR signaling pathway. Antibodies cross-reactive for both human and mice are used. 15-16 additional subtyping/functional markers are incorporated for the mouse and human panels. 8B-C, The phospho-specific antibody clones and the phospho-staining protocol are validated using two stimulation conditions, anti-CD3 and hydrogen peroxide (H₂O₂), in mouse splenocytes (8B) and Jurkat E6-1 cells (8C), leading to increased phosphorylation levels compared to the unstimulated condition. Stimulated-to-unstimulated ratios of arcsinh transformed phosphorylation levels are indicated by the color key. Red lines signify untested. 8D-E, Correlation matrices obtained from phospho-profiling CD8+ T cells from spleens of MC38-bearing WT mice (aggregate data from 4 mice) (8D) and peripheral blood (PB) of hepatocellular carcinoma (HCC) patients not exposed to immunotherapy (aggregate data from 4 patients) (8E). The correlation strengths are indicated by color (positive: red; negative: blue), the size of the circles, and annotated with numbers. Notable correlations are outlined. 8G, The result from the FlowSOM clustering into 25 metaclusters based on 14 lineage/functional markers and then annotated into 16 final subtypes of CD8+ T cells for phospho-profiling of the tumors is shown as a normalized metal intensity heatmap. Abbreviations: central memory subtype, CM; effector memory subtype, CM; effector subtype, Eff; “exhausted” subtype (positive expression of checkpoint markers), EX. 8H, Stacked bar graphs represent the CD8+ T cell profile upon clustering and annotation based on the results from the FlowSOM algorithm. 8I, Dimensionality reduction plot using UMAP.

FIGS. 9A-9D: Synthesis and Characterization of novel PTPN22 inhibitor, L-1. 9A, A quinolone derivative Core 1 with 3-Carboxy-4-quinolone scaffold which is commonly found in drugs and bioactive compounds could serve as a potential novel pTyr mimetic to engage the PTPN22 active site. To increase the potency and selectivity of Core 1 for PTPN22, we aimed to install molecular diversity to Core 1 in order to capture additional and less conserved interactions outside the pTyr-binding cleft (i.e. active site). Preliminary molecular modeling suggested that the carboxylic acid and the quinoline ring occupy the PTPN22 active site and the 6-position amino group is solvent exposed. The positioning of the 6-amino group affords us the opportunity to tether Core 1 to diverse molecular fragments in order to engage unique peripheral pockets in the vicinity of the PTPN22 active site. Based on preliminary modeling, we designed and synthesized a series of bivalent compounds by connecting the 6-amino group of Core 1, via a L-lysine linker, to 480 carboxylic acids that differ in size, polarity, solubility, and drug-like properties (R1). High throughput screening of the fragment-based focused library identified the biphenyl carboxylic acid group as one of the most potent fragments for PTPN22. To optimize the linker interaction with PTPN22, 16 different amino acids with neutral, acidic, and basic side chains (R2) were surveyed to further increase binding affinity, from which a novel PTPN22 inhibitor L-1 was identified. 9B, To determine the selectivity of L-1 for PTPN22, its inhibitory activity toward a panel of 16 mammalian PTPs were also measured. 9C, Kinetic analyses showing competitive inhibition of L-1 for PTPN22 with Ki=0.50±0.03 μM. 9D, In vivo pharmacokinetics data based on mass spectrometry quantification at 0 h, 1.0 h, 1.5 h, 2.0 h, 2.5 h, 3.0 h, 6.0 h, and 24.0 h from time of intraperitoneal injection of L-1 for three mice are shown.

FIGS. 10A-10E: L-1 augments in vivo antitumor immune responses. 10A-C, Immune profiles in MC38 tumors treated with vehicle (VEH) or L-1. Resulting UMAP plot (10A) from CyTOF analysis of the immune profile. Immune subsets (10B) and T cell subsets (10C) are represented as percentage of live cells. 10D-E, Given relatively low immune cell abundance in CT26 tumors, supervised gating analysis was performed to evaluate immune profiles in CT26 tumors treated with vehicle (VEH) or L-1. Mean+s.e.m., n=8, *p<0.05, **p<0.01, ***p<0.005, results of T-tests paired by batch. Abbreviations: checkpoint markers, Ckpt; dendritic cells, DC; double-negative T, DNT; granulocytic myeloid-derived suppressor cells/tumor associated neutrophils, G-MDSC/TAN; granzyme, Gz; monocytic myeloid-derived suppressor cells/myeloid cells, M-MDSC/MC; monocytes, MC; natural killer cells, NK; natural killer T cells, NKT; tumor-associated macrophages, TAM; cytotoxic T cells, Tc; helper T cells, Th; regulatory T cells, Treg.

FIG. 11: L-1 phenocopies PTPN22 KO functional marker expression profiles. Two separate experiments are shown: WT vs. PTPN22 KO and vehicle (VEH) vs. L-1. MC38 tumor-infiltrating immune cells are profiled by CyTOF. Mean metal intensities for each of the functional marker expression stratified by the cell types (n=8). Results from mixed linear modeling for differential analysis between the two groups are indicated by FDR-adjusted p values: #<0.2, *<0.1, ***<0.005.

FIGS. 12A-12E: A second small molecule inhibitor of PTPN22, LTV1, is tested against the MC38 tumor model. 12A, Treatment is given analogous to L1 as described previously. Inset: chemical structure of LTV1. Tumor growth curves are shown with mixed effect modeling result indicated as *p<0.05 (n=22-24). 12B-C, CyTOF profiling of the tumors resulted in abundance profiles of immune cell subtypes (12B) and functional marker expression profiles stratified by each of the cell subtype (12C). FDR-adjusted p value #<0.2, *<0.05, ***<0.005 (n=7). 12D, Splenocytes are incubated in LTV1 overnight. Mean±s.e.m. for MHCII-positive proportion in CD11b+, CD19+, or CD11c+ populations (n=6). Not significant (n.s.), *p<0.05, two-tailed unpaired t-tests comparing DMSO- and LTV1-treated groups. 12E, The effect of LTV1 on MC38 in vitro. Mean+/−SE bars (n=11-12, three independent experiments done in triplicates) are shown for each concentration.

FIG. 13: Abrogation of PTPN22 leads to significantly improved survival in the metastatic PDA model. In addition to subcutaneous models, WT or PTPN22 KO mice were injected with pancreatic ductal adenocarcinoma cell line (mutant Kras-Tp53 driven, “KPC”) intraportally via surgical hemispleen method and monitored for survival in the metastatic liver model. Kaplan-Meier curves are shown (N=9-10). ***p<0.005 log rank test.

FIG. 14: Antitumor efficacy of systemic PTPN22 abrogation is heavily contributed by T cells. WT or PTPN22 KO mice were inoculated with subcutaneous tumors (MC38) and treated with isotype antibodies or anti-CD4 or anti-CD8 antibodies to deplete the respective T cell compartments. Tumor volumes as measured by caliper (mean+SE) are shown (n=5-10 per arm).

FIG. 15: Abrogation of PTPN22 leads to enhanced clonal T cell response against tumors. T cells within MC38 tumors from WT, PTPN22 KO, WT vehicle-treated, and WT L1-treated mice were assayed for T cell receptor (TCR) repertoire using ImmunoSeq (Adaptive). Measures of TCR clonality and diversity (Shannon's Entropy) are compared as mean+SD. *p<0.05 by t-test (n=4-5 per group).

FIG. 16: PTPN22 inhibition synergizes with immune checkpoint inhibition. Mice were inoculated with subcutaneous tumors (MC38 or CT26) and treated with negative control agents (vehicle formulation “VEH” or isotype antibodies “ISO”), PTPN22 inhibitor “L1” alone, anti-PD1 antibody alone “PD1”, or both. Tumor volumes were measured by calipers and mean+/−SE are shown (n=9-10 per arm).

FIG. 17: Schematic diagram of selection criteria for case control study of patients having the rs2476601 PTPN22 variant.

FIG. 18: Table 2: Mouse Tumor Immune Profiling Panel.

FIG. 19: Table 3: Mouse Phospho-Profiling Panel.

FIG. 20: Table 4: Human Phospho-Profiling Panel.

FIG. 21: Table 5: Barcoding and Ancillary Markers.

FIG. 22: Table 6: Flow Cytometry Antibodies.

### DETAILED DESCRIPTION OF THE INVENTION

The described invention is distinctly different from a prior publication demonstrating that T cells with existing PTPN22 KO can be adoptively transferred to control the growth of tumors (23). It differs because this invention for the first time shows it is possible to systemically inhibit PTPN22 in immune cells of a subject, such as T-cells, to enhance endogenous T cell killing of tumor cells in vivo. This invention is also distinct but complementary to existing immune checkpoint antibody therapies because this approach inhibits a different class of autoimmune T cell regulatory proteins—non-receptor protein tyrosine phosphatases; changes in phosphorylation states within the T cell receptor signaling rather than ligand binding are able to regulate T cell function..

Therefore, in accordance with a first embodiment, the present invention provides a method of treating cancer in a subject comprising administering to the subject an effective amount of an inhibitor of PTPN22.

In accordance with a second embodiment, the present invention provides a method of treating cancer in a subject comprising administering to the subject an effective amount of an inhibitor of PTPN22 comprising the compound LTV1, or a salt, solvate or stereoisomer thereof.

By “LTV1” is meant the compound with the chemical structure of compound 1 containing a thiobarbituric acid scaffold, that selectively inhibits PTPN22:

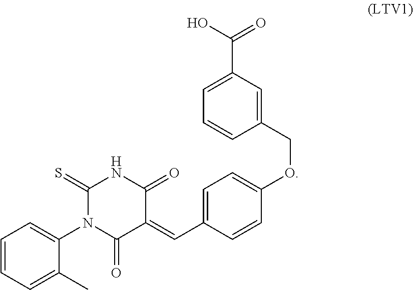 [SMILES: Cc1ccccc1N1C(=O)/C(=C/c2ccc(OCc3cccc(C(=O)O)c3)cc2)C(=O)NC1=S]

In accordance with a third embodiment, the present invention provides a method of treating cancer in a subject comprising administering to the subject an effective amount of an inhibitor of PTPN22 comprising compounds of Formulas (I), (II), (III), and (IV).

A compound of Formula (I) comprises:

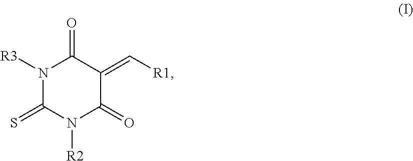

wherein R₁ is selected from the group consisting of

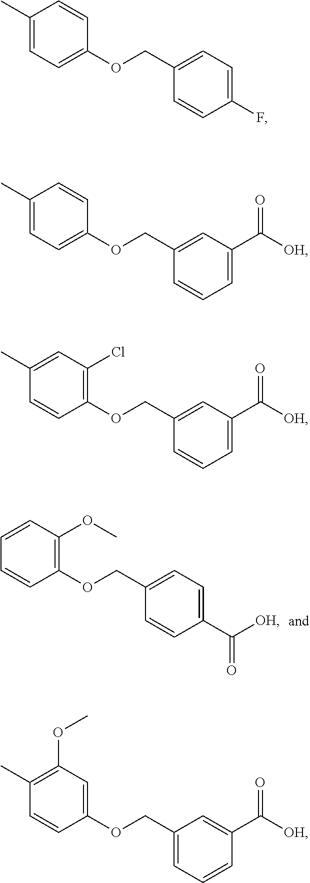

wherein R₂ and R₃ are the same or different and are each selected from the group consisting of H, CH₃,

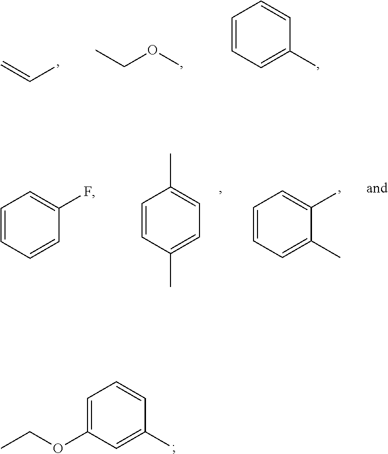

or a pharmaceutically acceptable salt, solvate, or stereoisomer thereof.

A compound of Formula (II) comprises:

[symbol]

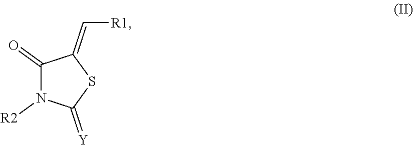

wherein R₁ is selected from the group consisting of H, CH₃,

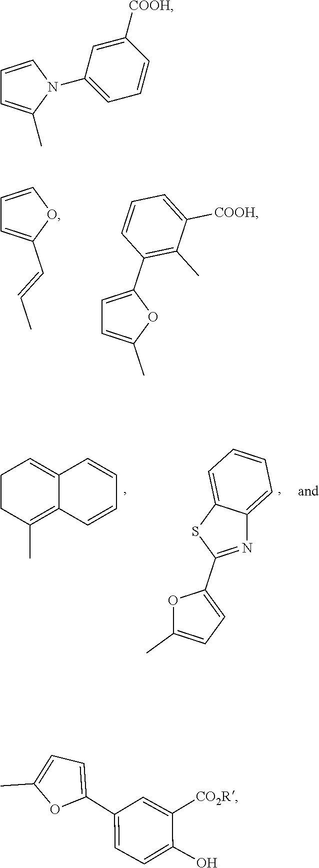

wherein R₂ is selected from the group consisting of H, CH₃,

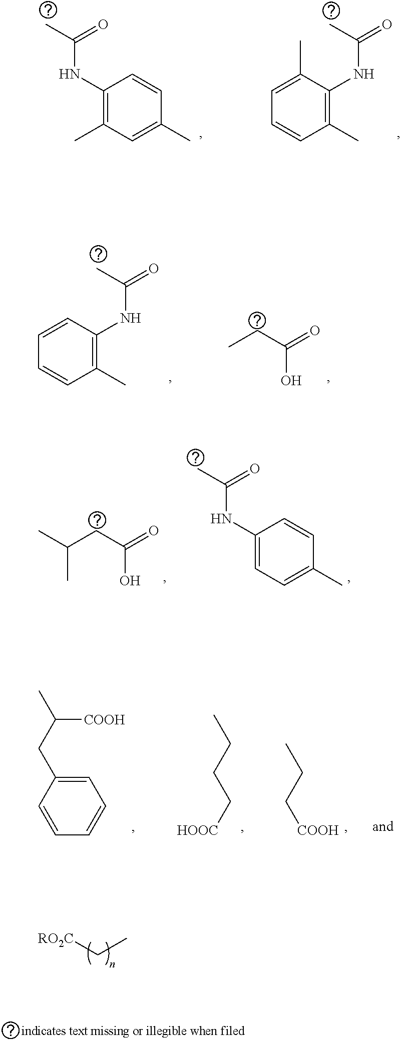

wherein N is 1 to 20, and Y is S or O, or a pharmaceutically acceptable salt, solvate, or stereoisomer thereof.

A compound of Formula (III) comprising:

R₃P—Au—Cl,Au(I), (III),

[symbol]

wherein R is selected from the group consisting of

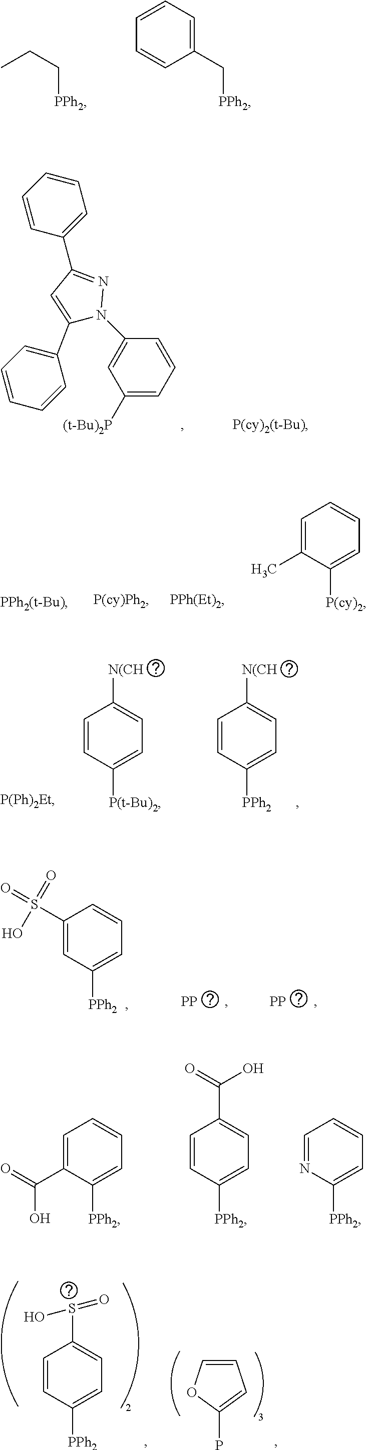

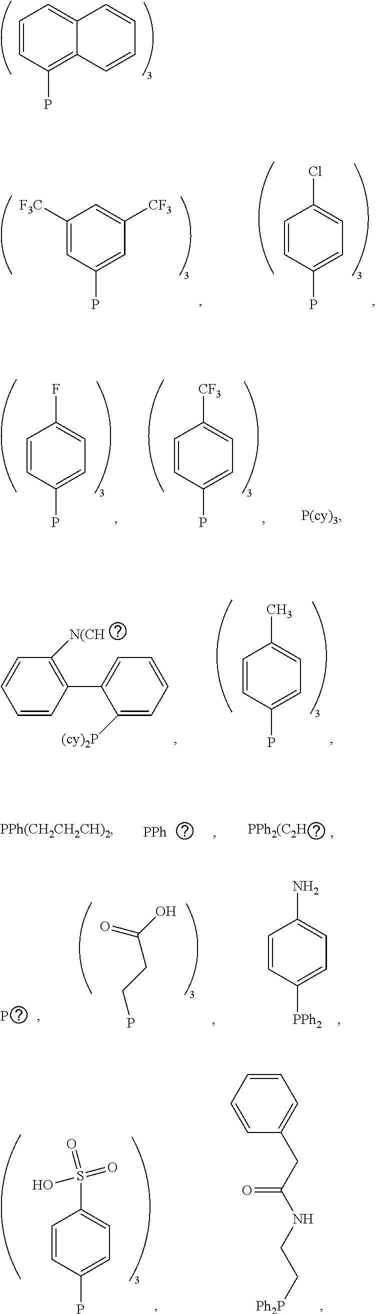

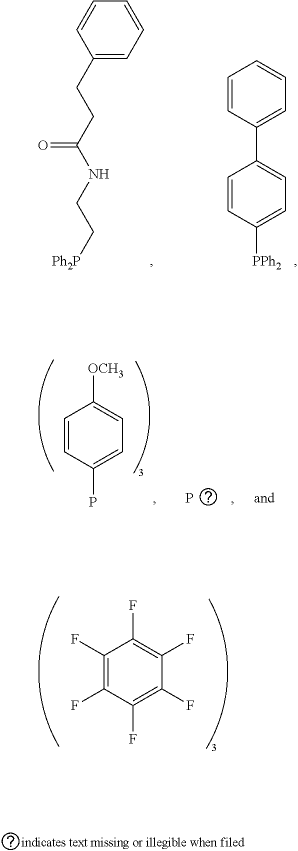

or a pharmaceutically acceptable salt, solvate, or stereoisomer thereof.

A compound of Formula (IV) comprising:

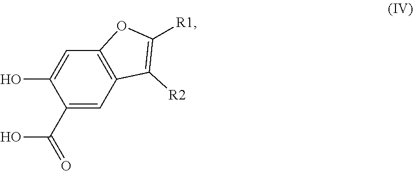

wherein R₁ is selected from the group consisting of

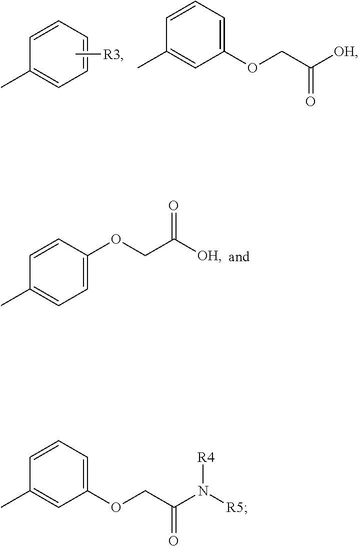

wherein R₂ is selected from the group consisting of

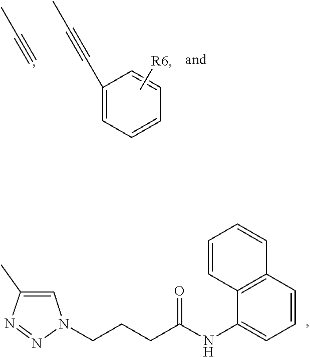

and wherein R₃=4-OCF₃,

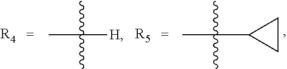

R₆=3-Cl; or a combination of these moieties thereof.

In accordance with a fourth embodiment, the present invention provides a method of treating cancer in a subject comprising administering to the subject an effective amount of an inhibitor of PTPN22 comprising the compound L1, or a salt, solvate or stereoisomer thereof.

By “L-1” is meant a compound of formula V:

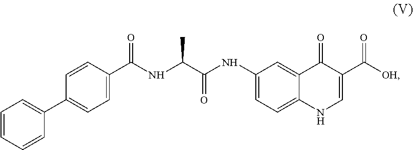 [SMILES: C[C@H](NC(=O)c1ccc(-c2ccccc2)cc1)C(=O)Nc1ccc2[nH]cc(C(=O)O)c(=O)c2c1]

or a salt, solvate or stereoisomer thereof which is an inhibitor of PTPN22.

In accordance with a fifth embodiment, the present invention provides a method of treating cancer in a subject comprising administering to the subject an effective amount of an inhibitor of PTPN22 and an effective amount of at least one additional anti-cancer agent.

It will be understood to those of skill in the art that the term “therapeutic agent” is any agent capable of affecting the structure or function of the body of a subject or is an agent useful for the treatment or modulation of a disease or condition in a subject suffering therefrom. Examples of therapeutic agents can include any drugs known in the art for treatment of disease indications.

An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se, as well as pharmaceutically acceptable, pharmacologically active salts, esters, amides, prodrugs, metabolites, analogs etc.

Specific examples of useful biologically active agents the above categories include: anti-cancer and anti-neoplastics such as androgen inhibitors, antimetabolites, cytotoxic agents, and immunomodulators. Antineoplastic agents can include, for example, alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics. Further examples can include alkylating antineoplastic agents, such as carboplatin and cisplatin; nitrosourea alkylating antineoplastic agents, such as carmustine (BCNU); antimetabolite antineoplastic agents, such as methotrexate; pyrimidine analog antineoplastic agents, such as fluorouracil (5-FU) and gemcitabine; hormonal antineoplastics, such as goserelin, leuprolide, and tamoxifen; natural antineoplastics, such as aldesleukin, interleukin-2, docetaxel, etoposide, interferon; paclitaxel, other taxane derivatives, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine.

In a further embodiment, the compositions and methods of the present invention can be used in combination with one or more additional therapeutically active agents which are known to be capable of treating conditions or diseases discussed above. For example, the compositions of the present invention could be used in combination with one or more known therapeutically active agents, to treat a proliferative disease such as a tumor or cancer. Non-limiting examples of other therapeutically active agents that can be readily combined in a pharmaceutical composition with the compositions and methods of the present invention are enzymatic nucleic acid molecules, allosteric nucleic acid molecules, antisense, decoy, or aptamer nucleic acid molecules, antibodies such as monoclonal antibodies, small molecules, and other organic and/or inorganic compounds including metals, salts and ions.

In accordance with a sixth embodiment, the present invention provides a method of treating cancer in a subject comprising administering to the subject an effective amount of an inhibitor of PTPN22 and an effective amount of at least one checkpoint inhibitor.

Examples of anti-cancer agents that may be used in the present invention include immune checkpoint inhibitors such as: a PD1 inhibitor, a PDL1 inhibitor; a CTLA4 inhibitor; a LAG3 inhibitor; a TIGIT inhibitor; IDO inhibitor; a co-stimulatory agonist therapy; a cancer vaccine; a T cell transfer therapy, and a combination thereof. A PD1 inhibitor, PDL1 inhibitor, or a CTLA4 inhibitor maybe an antibody. Examples of PD1 inhibitors and the PDL1 inhibitors used in the present invention include pembrolizumab, nivolumab, durvalumab, atezolizumab, avelumab, and a combination thereof. Examples of a CTLA4 inhibitors used in the present invention include ipilimumab, tremelimumab, and a combination thereof. Examples of co-stimulatory agonist therapies used in the present invention include anti-OX40 therapy, anti-CD40 therapy, anti-41BB therapy, and a combination thereof.

In some embodiments, anticancer agents may be administered before, after, or concurrently with a systemic inhibitor of PTPN22.

Suitable systemic inhibitors of PTPN22 used in the present invention include PTPN22 antagonists, PTPN22 gene suppressors, or a combination thereof.

Included within the compounds of the present invention are the tautomeric forms of the disclosed compounds, isomeric forms including diastereoisomers, and the pharmaceutically-acceptable salts thereof. The term “pharmaceutically acceptable salts” embraces salts commonly used to form alkali metal salts and to form addition salts of free acids or free bases. Examples of acids which may be employed to form pharmaceutically acceptable acid addition salts include such inorganic acids as hydrochloric acid, sulphuric acid and phosphoric acid, and such organic acids as maleic acid, succinic acid and citric acid. Other pharmaceutically acceptable salts include salts with alkali metals or alkaline earth metals, such as sodium, potassium, calcium and magnesium, or with organic bases, such as dicyclohexylamine. Suitable pharmaceutically acceptable salts of the compounds of the present invention include, for example, acid addition salts which may, for example, be formed by mixing a solution of the compound according to the invention with a solution of a pharmaceutically acceptable acid, such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. All of these salts may be prepared by conventional means by reacting, for example, the appropriate acid or base with the corresponding compounds of the present invention.

Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicines, the salts of the compounds of the present invention should be pharmaceutically acceptable salts. Other salts may, however, be useful in the preparation of the compounds according to the invention or of their pharmaceutically acceptable salts.

In addition, embodiments of the invention include hydrates of the compounds of the present invention. The term “hydrate” includes but is not limited to hemihydrate, monohydrate, dihydrate, trihydrate and the like. Hydrates of the compounds of the present invention may be prepared by contacting the compounds with water under suitable conditions to produce the hydrate of choice.

With respect to the pharmaceutical compositions described herein, the carrier can be any of those conventionally used, and is limited only by physico-chemical considerations, such as solubility and lack of reactivity with the active compound(s), and by the route of administration. The carriers described herein, for example, vehicles, adjuvants, excipients, and diluents, are well-known to those skilled in the art and are readily available to the public. It is preferred that the carrier be one which is chemically inert to the active agent(s), and one which has little or no detrimental side effects or toxicity under the conditions of use. Examples of the carriers include solid compositions such as solid-state carriers or latex beads.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

The choice of carrier will be determined, in part, by the particular pharmaceutical composition, as well as by the particular method used to administer the composition. Accordingly, there are a variety of suitable formulations of the pharmaceutical composition of the invention.

In accordance with some embodiments, a systemic inhibitor of PTPN22 is a PTPN22 gene suppressor such as one or more siRNAs, one or more CRISPR elements, as examples. For example, a PTPN22 gene suppressor maybe a PTPN22 siRNA. In some embodiments, the PTPN22 gene suppressor is expressed from a vector that, when administered to the subject, integrates into one or more cells of the subject.

In accordance with some embodiments, examples of solid cancers treated by the methods of the present invention include anal, bladder, bone, breast, central nervous system, cervical, colon, endometrial, esophageal, gastric, head and neck, hepatobiliary, kidney, leukemia, lung, lymphoma, melanoma, merkel cell, ovarian, pancreatic, prostate, soft tissue sarcomas, testicular, thymoma, thyroid, uterine, and a combination thereof. The solid cancer maybe metastatic. Examples of hematological cancers treated by the methods of the present invention include: acute myeloid leukemia, acute lymphoblastic leukemia, chronic myeloid leukemia, chronic lymphocytic leukemia, and multiple myeloma.

In some embodiments, a pharmaceutical composition comprising the systemic inhibitor of PTPN22 is administered to the subject. In some embodiments, the pharmaceutical composition further comprises a neoadjuvant, an adjuvant, or a combination thereof.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “activity” refers to the ability of a gene to perform its function such as protein tyrosine phosphatase, non-receptor type 22 (PTPN22) affecting the responsiveness of T and B cell receptors.

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind, for example, PD-L1, specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)₂ fragment in which the two arms of the antibody molecule remain linked and comprise two-antigen binding sites. The F(ab′)₂ fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “disease” is meant any condition or disorder that damages or interferes with the normal function of a cell, tissue, or organ. Examples of diseases include cancer.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “express” refers to the ability of a gene to express the gene product including for example its corresponding mRNA or protein sequence (s).

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “gene suppression” is meant the regulation of gene expression in a cell to prevent the or reduce the expression of a certain gene. Gene suppression may occur during either transcription or translation. Gene silencing is often considered the same as gene knockdown. When genes are silenced, their expression is reduced. In contrast, when genes are knocked out, they are completely erased from the organism's genome, and, thus, have no expression. Examples of gene expression methods include RNAi, CRISPR, or siRNA.

By “gene suppressor” is meant an agent that causes gene suppression such as RNAi, CRISPR vectors, or SiRNA as examples.

By “immune cells” is meant cells of the immune system that can be categorized as lymphocytes (T-cells, B-cells and NK cells), neutrophils, and monocytes/macrophages.

The term, “obtaining” as in “obtaining an agent” includes synthesizing, purchasing, or otherwise acquiring the agent.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

The term “PD-1” refers to program death receptor-1. The term “anti PD-1 refers to one or more entity, such as an antibody for example, that bind to PD-1 and modulate its activity, preferably inhibit its activity.

The term “PD-L1” refers to one of the major ligands of PD-1 called program death ligand 1. The term “anti PD-L1” refers to one or more entity, such as an antibody for example, that bind to PD-L1.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

By “prevent,” “preventing,” “prevention,” “prophylactic treatment” is meant reducing the probability of developing a disorder or condition in a subject, who does not have, but is at risk of or susceptible to developing a disorder or condition.

By “PTPN22”, “LYP”, “LYP1”, “LYP2”, “PEP”, “PTPN8”, “PTPN22.6”, or “PTPN22.5” is meant a gene and protein thereof that affects the responsiveness of T and B cell receptors, and mutations are associated with increases or decreases in risks of autoimmune diseases. The gene encodes a protein tyrosine phosphatase which is expressed primarily in lymphoid tissues. This enzyme is involved in several signaling pathways associated with the immune response. Based on models of the murine phosphatase, structural identification, and human genetics, the phosphatase forms complexes with C-src tyrosine kinase (Csk), associated with the control of Src family members. The mutation Arg620Trp disrupts binding to Csk, alters the responsiveness of T and B cell receptors, and is associated with autoimmune diseases.

By “PTPN22 single-nucleotide polymorphism (SNP), rs2476601” means has a C->T variant C1858T and results in an arginine (R) to tryptophan (W) amino acid change at amino acid position 620. It has been associated with subjects that have rheumatoid arthritis and Type 1 Diabetes.

In accordance with an embodiment, the present invention provides for identifying a subject as having T-Cells which have reduced expression of PTPN22 compared to control expression levels comprising, obtaining a DNA sample from the subject, and sequencing the DNA of the sample for the presence of the T genotype of the functional single nucleotide polymorphism rs2476601 in the PTPN22 gene.

In some embodiments, the companion diagnostic test may comprise: a) obtaining a biological sample from a subject that is undergoing a treatment or is considered for a treatment for cancer; b) isolating genomic DNA from said biological sample; c) assaying the panel of biomarkers; d) generating an output with a computer algorithm based on the assay results of said panel of biomarkers; and/or e) determining the likely responsiveness of said subject to said treatment. In some embodiments, the SNPs may be assayed by sequencing, capillary electrophoresis, mass spectrometry, single-strand conformation polymorphism (SSCP), electrochemical analysis, denaturing HPLC and gel electrophoresis, restriction fragment length polymorphism, hybridization analysis, single-base extension, and/or microarray.

In accordance with an embodiment, the present invention provides for identifying a subject as having a reduced risk of cancer comprising obtaining a DNA sample from the subject, and sequencing the DNA of the sample for the presence of the T genotype of the functional single nucleotide polymorphism rs2476601 in the PTPN22 gene.

The present invention describes a genomic biomarker that have been discovered to correlate with different responses (efficacy, adverse effect, and other end points) among patients receiving treatment regime including anticancer agents, in treating diseases such as, cell lung cancer. The biomarker can be used in companion dosgnostic tests which can help to predict drug responses and apply drugs only to those who will be benefited, or exclude those who might have adverse effects, by the treatment.

The term “biomarker” or “marker” as used herein refers generally to a molecule, including a gene, protein, carbohydrate structure, or glycolipid, the expression of which in or on a mammalian tissue or cell or secreted can be detected by known methods (or methods disclosed herein) and is predictive or can be used to predict (or aid prediction) for a mammalian cell's or tissue's sensitivity to, and in some embodiments, to predict (or aid prediction) an individual's responsiveness to treatment regimens.

As used herein, a “pharmacogenomic biomarker” is an objective biomarker which correlates with a specific clinical drug response or susceptibility in a subject (see, e.g., McLeod et al., Eur. J. Cancer (1999) 35:1650-1652). It may be a biochemical biomarker, or a clinical sign or symptom. The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of DNA, RNA, or protein for specific tumor markers in a subject, a drug or course of treatment may be selected that is optimized for the treatment of the specific tumor likely to be present in the subject. Similarly, the presence or absence of a specific sequence mutation or polymorphism may correlate with drug response. The use of pharmacogenomic biomarkers therefore permits the application of the most appropriate treatment for each subject without having to administer the therapy.

As used herein, the term “polymorphic locus” refers to a region in a nucleic acid at which two or more alternative nucleotide sequences are observed in a significant number of nucleic acid samples from a population of individuals. A polymorphic locus may be a nucleotide sequence of two or more nucleotides, an inserted nucleotide or nucleotide sequence, a deleted nucleotide or nucleotide sequence, or a microsatellite, for example. A polymorphic locus that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic locus is often one nucleotide in length, which is referred to herein as a “single nucleotide polymorphism” or a “SNP.” In some embodiments, the high-density genotyping may be conducted by using SNPs. In some embodiments, about 1,000-5,000,000 or more SNPs, may be used. In some embodiments, the high-density genotyping may be array-based. In some embodiments, the high-density genotyping may be conducted by using sequencing, such as high-throughput sequencing.

Where there are two, three, or four alternative nucleotide sequences at a polymorphic locus, each nucleotide sequence is referred to as a “polymorphic variant” or “nucleic acid variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele.

In genetic analysis that identifies one or more pharmacogenomic biomarkers, samples from individuals having different values in a relevant phenotype often are allelotyped and/or genotyped. The term “allelotype” as used herein refers to a process for determining the allele frequency for a polymorphic variant in pooled DNA samples from cases and controls. By pooling DNA from each group, an allele frequency for each locus in each group is calculated. These allele frequencies are then compared to one another.

A genotype or polymorphic variant may be expressed in terms of a “haplotype,” which as used herein refers to a set of DNA variations, or polymorphisms, that tend to be inherited together. A haplotype can refer to a combination of alleles or to a set of SNPs found on the same chromosome. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

The term “sample”, as used herein, refers to a composition that is obtained or derived from a subject of interest that contains a cellular and/or other molecular entity that is to be characterized and/or identified, for example based on physical, biochemical, chemical and/or physiological characteristics. For example, the phrase “clinical sample” or “disease sample” and variations thereof refer to any sample obtained from a subject of interest that would be expected or is known to contain the cellular and/or molecular entity that is to be characterized.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR 2 (“amidate”), P(O)R, P(O)OR′, CO or CH 2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, the term “phenotype” refers to a trait which can be compared between individuals, such as presence or absence of a condition, a visually observable difference in appearance between individuals, metabolic variations, physiological variations, variations in the function of biological molecules, and the like. A phenotype can be qualitative or quantitative. An example of a phenotype is responsiveness to a treatment, such as a drug.

The term “prediction” or “prognosis” is used herein to refer to the likelihood that a patient will respond either favorably or unfavorably to a drug or set of drugs. In one embodiment, the prediction relates to the extent of those responses. In one embodiment, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc.

The present invention describes a genomic biomarker of a SNP in the PTPN22 gene that correlates with the improved activity of immune checkpoint inhibitor, such as such as a PDL-1 inhibitor. These biomarkers can be used to identify the patients who are most likely to benefit or experience adverse effect from immune checkpoint inhibitor treatment.

Generally, an isolated SNP-containing nucleic acid molecule comprises one or more SNP positions disclosed by the present invention with flanking nucleotide sequences on either side of the SNP positions. A flanking sequence can include nucleotide residues that are naturally associated with the SNP site and/or heterologous nucleotide sequences. Preferably the flanking sequence is up to about 500, 300, 100, 60, 50, 30, 25, 20, 15, 10, 8, or 4 nucleotides (or any other length in-between) on either side of a SNP position, or as long as the full-length gene or entire protein-coding sequence (or any portion thereof such as an exon).

By “ranges” is meant all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reduces” is meant a negative alteration for example by at least 10%, 25%, 50%, 75%, or 100% or by at least 2, 5, 10, 25, or 50 fold. For example, regarding gene suppressor a gene's transcription and or translation may be reduced by at least 10%, 25%, 50%, 75%, or 100% or by at least 2, 5, 10, 25, or 50 fold.

A “reference” refers to a standard or control conditions such as a sample (human cells) or a subject that is a free, or substantially free, of an agent such as one or more antagonist of PTPN22.

By “solid cancer” or “solid tumor” is meant an abnormal mass of tissue that are malignant. Different types of solid tumors are named for the type of cells that form them. Examples of solid cancers are adenocarcinomas, sarcomas, carcinomas, and lymphomas.

By “specifically binds” is meant a compound or antibody that recognizes and binds a nucleic acid, protein or peptide such as those related to PTPN22, but which does not substantially recognize and bind other molecules in a sample, for example, a biological sample, which naturally includes a nucleic acid, protein, or polypeptide of PTPN22.

By “subject” is meant any individual or patient to which the method described herein is performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

By “systemic” is meant relating to, spread throughout, or having effect on a system (the whole) such as a human body, for example, as opposed to a particular part.

By “systemic inhibition” is meant targeting a protein or a gene by way of administering an agent directly into the entire system or the body by oral, subcutaneous, intraperitoneal, or intravenous routes.

By “treat,” treating,” “treatment,” is meant to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

Such treatment (surgery and/or chemotherapy) will be suitably administered to subjects, particularly humans, suffering from, having, susceptible to, or at risk for pancreatic cancer or disease, disorder, or symptom thereof. Determination of those subjects “at risk” can be made by any objective or subjective determination by a diagnostic test or opinion of a subject or health care provider (e.g., genetic test, enzyme or protein marker, a marker (as defined herein), family history, and the like).

### Pharmaceutical Preparations

Pharmaceutical compositions of the present invention comprise an effective amount of one or more suppressors of expression PTPN22 and/or one or more antagonist of PTPN22 activity such as LTV1, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one or more suppressors of expression PTPN22 and/or one or more antagonist of PTPN22 (or additional active ingredients) will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The one or more suppressors of expression PTPN22 and/or one or more antagonist of PTPN22 may comprise different types of carriers depending on whether it is to be administered in solid, liquid or aerosol form, and whether it need to be sterile for such routes of administration as injection. The present compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, id.).

The one or more suppressors of expression PTPN22 and/or one or more antagonist of PTPN22 may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Furthermore, in accordance with the present disclosure, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include one or more suppressors of expression PTPN22 and/or one or more antagonist of PTPN22, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the one or more suppressors of expression PTPN22 and/or one or more antagonist of PTPN22 may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound, such as L-1 or LTV-1. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Alimentary Compositions and Formulations

In some embodiments of the present disclosure, the one or more suppressors of expression PTPN22 and/or one or more antagonist of PTPN22 activity are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### Parenteral Compositions and Formulations

In further embodiments, one or more suppressors of expression PTPN22 and/or one or more antagonist of PTPN22 activity may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences,” id.). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### Miscellaneous Pharmaceutical Compositions and Formulations

In other preferred embodiments of the invention, the active compound, such as one or more suppressors of expression PTPN22 and/or one or more antagonist of PTPN22 activity, may be formulated for administration via various miscellaneous routes, for example, topical (i.e., transdermal) administration, mucosal administration (intranasal, vaginal, etc.) and/or inhalation. Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and laurocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### Kits of the Disclosure

Any of the compositions described herein may be comprised in a kit. In a non-limiting example, one or more suppressors of expression PTPN22 and/or one or more antagonist of PTPN22 activity (for example, LTV1) may be comprised in a kit. The kits may comprise a suitably aliquoted one or more suppressors of expression PTPN22 and/or one or more antagonist of PTPN22 activity and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing one or more suppressors of expression PTPN22 and/or one or more antagonist of PTPN22 and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The one or more suppressors of expression PTPN22 and/or one or more antagonist of PTPN22 composition(s) may be formulated into an injectable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLES

Cell Lines

MC38 (Kerafast) were cultured in DMEM supplemented with 1% L-glutamine containing 10% FBS, 0.1 mM non-essential amino acids, 1 mM sodium pyruvate, 100U/mL penicillin/streptomycin, and 10 mM HEPES (Life Technologies). EG7 (ATCC) and Jurkat E6-1 (ATCC) were cultured in RPMI supplemented with glutamine containing 10% FBS, 100 U/mL penicillin/streptomycin. Culture medium for EG7 also contained 10 mM HEPES and 400 μg/ml geneticin G418. Cell line authentication by STR DNA profiling was done at the Johns Hopkins Genetic Resources Core Facility. All cell lines were tested for mycoplasma and were negative.

Animal Models

All mice husbandry, experiments, and euthanasia were performed in accordance with Johns Hopkins IACUC approved protocols. WT C57Bl/6J, Balb/cJ, and PTPN22 KO (B6.Cg-Ptpn22tm2Achn/J) mice were purchased from JAX and bred in-house. For syngeneic immunocompetent mouse models, aged-matched 8-12 week old female mice were injected with MC38 (2.5×10⁵) or EG7 (2.5×10⁵ for rejection and 5×10⁵ for treatment) or CT26 (5×10⁵) subcutaneously in the right hind limb. PD1 inhibition therapy were performed with 200 μg/mouse anti-PD1 antibody (clone: RMP1-14, BioXCell) and compared against matched isotype (clone: 2A3, BioXCell). For L1 treatment experiments, L1 was dissolved in DMSO and was diluted to 10% (v/v) DMSO and 5% (v/v) cremophor-EL (Sigma) in PBS to be administered at 10 mg/kg/dose. LTV1 was also dissolved in DMSO and diluted to the same final formulation for administration at 6 mg/kg/dose. Vehicle injections contained 10% DMSO and 5% cremophor-EL in PBS.

Human Peripheral Blood Samples

The evaluation of banked peripheral blood mononuclear cells (PBMCs) was performed in accordance with the protocols approved by the Johns Hopkins Institutional Review Board (IRB). All peripheral blood specimens were obtained with written patient consent (IRB number CIR00051274). Baseline samples from four de-identified patients with hepatocellular carcinoma treated at the Sidney Kimmel Comprehensive Cancer Center at Johns Hopkins were identified from the Johns Hopkins Liver Cancer biobank. Blood was collected in two BD Vacutainer CPT—Cell Preparation Tube with Sodium Heparin (BD Biosciences) and processed within two hours of collection. Tubes were centrifuged at room temperature for approximately thirty minutes at 1800 Relative Centrifugal Force (RCF). PBMCs were aspirated and pooled into a separate 50 mL conical and washed with RPMI medium. PBMCs were then counted and resuspended in AIMS and 10% DMSO at a concentration of five million cells per vial. Cryovials were initially stored at −80° C. and transferred to liquid nitrogen for long term storage prior to staining.

PheWAS and BioVU Database Analyses

Description and methods related to BioVU database is provided on this website: victr.vumc.org/biovu-description/. The allele frequencies and association analysis of rs2476601 in the PheWAS_GWAS table were calculated using MEGA (Multi-Ethnic Genotyping Array) data.

Patient Cohort Study Using BioVU Database.

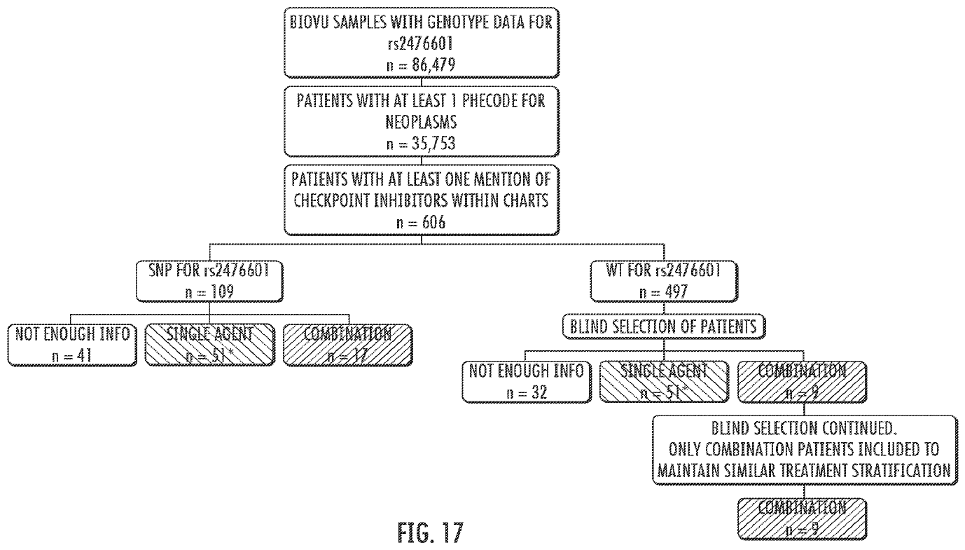

A retrospective case-controlled study using Electronic Health Records (EHR) within the Vanderbilt University Medical Center “Synthetic Derivative” (SD) and BioVU database was carried out to determine the utility of the PTPN22 SNP variant Rs2476601 as a predictive biomarker for response to single agent immunotherapy. The SD is a de-identified copy of the main hospital medical record databases created for research purposes. The de-identification of SD records was achieved primarily through the application of a commercial electronic program, which was applied and assessed for acceptable effectiveness in scrubbing identifiers. The SD database (which contains over 1.5 million electronic records, with no defined exclusions) was accessed through database queries and search queries based on patient inclusion criteria. Genetic data is integrated with the SD through the BioVU database. The primary outcome of the study was progression free survival (PFS). Eligible patients were selected based on the availability of genotype data for rs2476601 within the BioVu database (n=86,479). Patients selected for the study contained at least one PheCode (phenotypic codes used in SD) for neoplasm, regardless of tissue type, and with mention of at least one of the following immunotherapy checkpoint inhibitors; Ipilimumab, Pembrolizumab, Nivolumab, Atezolizumab, Durvalumab (n=606), within their EHR. There was no restriction regarding age, race, ethnicity, or gender. Eligible patients for case selection contained the rs2476601 PTPN22 variant and may be heterozygous or homozygous for the variant. Patients were stratified for single agent (n=51) or combination therapy (n=17). Patients that did not contain the rs2476601 PTPN22 variant or any other alteration within the PTPN22 gene were used as controls. Due to the significantly larger sample size of the WT control arm, a case-control study design was implemented to maintain equal stratification of single agent and combination therapy cases across both arms. Blind selection of control patients was carried out and case-matched. Within the control arm, 92 patients were blindly selected and screened to obtain 51 single agent therapy cases, 9 combination therapy cases, and 32 cases with not enough information. To maintain equivalent sample sizes across treatment arms, an additional 52 patients were blind selected and screened for cases of combination therapy only (n=9). This is summarized in FIG. 17. Retrospective chart analysis was used to determine PFS from start of specified immunotherapy to time of progression based on scan dates and physician remarks. Statistical significance was determined using the log-rank (Mantel-Cox) test via GraphPad Prism 8.0 Software.TCGA

RNA sequencing (RNAseq) Level 3 RSEM normalized data from TCGA were accessed from the Broad Institute TCGA GDAC Firehose (ezid.cdlib.org/id/doi:10.7908/C16W9975) and log₂-transformed. As a measure of mutational burden, we used the log₂-transformed number of non-synonimus mutations per sample from the TCGA MC3 project34. CIBERSORT scores for the TCGA samples were accessed from Thorsson et al. 35, and xCell scores were downloaded from the xCell web site (xcell.ucsfedu/). The Spearman's correlation coefficients were reported for correlation of PTPN22 expression and CIBERSORT or xCell scores and mutational burden. The Pearson's correlation coefficients we reported for correlation of expression of PTPN22 and immune regulation markers. Univariate survival analyses were generated from The Cancer Immunome Atlas (tcia.at/home)³⁶.

Pharmacology

Synthesis and Characterization of Novel Inhibitor of PTPN22, L-1

General Synthetic Procedures and Reagents.

Unless otherwise specified, all reagents were purchased from commercial suppliers and used directly without further purification. Analytical thin layer chromatography (TLC) was performed on 0.25 mm silica gel 60-F254. Column chromatography was performed using KP-SIL silica gel (Biotage, USA), and flash column chromatography was performed on Biotage prepacked columns using the automated flash chromatography system Biotage Isolera One. The ¹H and ¹³C NMR spectra were recorded on a Bruker AVANCE 500 MHz instrument. Chemical shifts for Proton magnetic resonance spectra (1H NMR) were quoted in parts per million (ppm) referenced to the appropriate solvent peak or 0.0 ppm for tetramethylsilane (TMS). The following abbreviations were used to describe peak splitting patterns when appropriate: br=broad, s=singlet, d=doublet, t=triplet, q=quartet, m=multriplet, dd=doublet of doublet. Coupling constants, J, were reported in hertz unit (Hz). Chemical shifts for ¹³C NMR were reported in ppm referenced to the center line at 39.52 of DMSO-d. Low-resolution mass spectra and purity data were obtained using an Agilent Technologies 6470 series, triple quadrupole LC/MS. High-resolution mass spectra (HRMS) were recorded on an Agilent Mass spectrometer using ESI-TOF (electrospray ionization-time of flight).

Synthesis of Compound L-1.

Starting from p-nitroaniline (2) and diethyl ethoxymethylenemalonate (3), advanced intermediate ethyl 6-nitro-4-oxo-1,4-dihydroquinoline-3-carboxylate (4) was obtained following a previously reported protocol1. The intermediate was then hydrogenated by Pd/C and H2 to provided compound (5) in good yield. Condensation of compound (5) with Fmoc-L-Ala-OH in the presence of HOBt, HBTU and DIPEA in DMF gave the Fmoc-protected intermediate (6). Deprotection of the Fmoc group was carried by using 20% piperidine containing DMF solution at room temperature. Compound (8) was obtained by condensing compound (7) with biphenyl-4-carboxylic acid in similar reaction condition as the previous condensation reaction. Final hydrolyzation using aq KOH in a mixture of MeOH/H2O afforded high yield of L-1 as free acid after acidification by aq HCl.

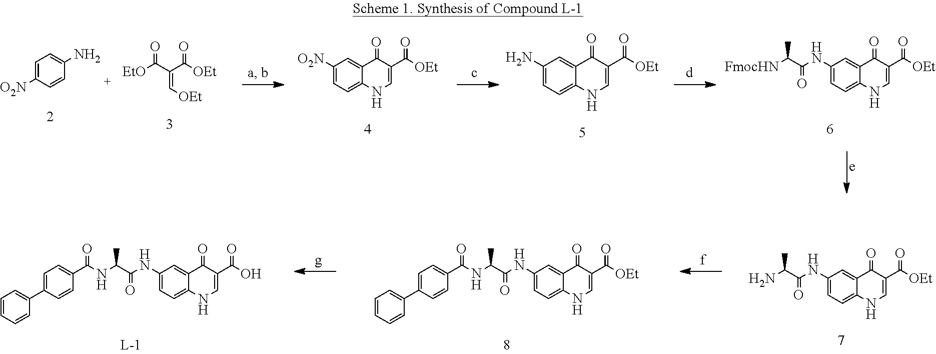

Reagents and conditions: (a) 135° C., 2 h, 75%; (b) PhOPh, reflux, 1 h, 95%; (c) Pd/C, H2, DMF, 100° C., 12 h, 85%; (d) Fmoc-L-Ala-OH, HOBt, HBTU, DIPEA, DMF, r.t., overnight, 65%; (e) Piperidine, DMF, r.t., 1 h, 69%; (0 Biphenyl-4-carboxylic acid, HOBt, HBTU, DIPEA, DMF, r.t., overnight, 78%; (g) KOH, MeOH/H2O, 60° C., 16 h, 90%.

### 6-amino-4-oxo-1,4-dihydroquinoline-3-carboxylic Acid (Core 1)

Purchased from Princeton BioMolecular Research, Inc. as pale yellow solid. ¹H NMR (500 MHz, DMSO) δ 15.99 (s, ¹H), 13.12 (brs, ¹H), 8.60 (s, ¹H), 7.55 (d, J=8.8 Hz, ¹H), 7.32 (d, J=2.6 Hz, ¹H), 7.18 (dd, J=2.6, 8.8 Hz, ¹H), 5.79 (s, ¹H); LC-MS (ESI): 205.0 (M+H)+, 203.0 (M−H)−; HRMS (ESI-TOF): m/z [M−H]− calculated for C10H7N2O3: 203.0462, found: 203.0461; Purity: >95% (UV, 2\, =254 nm).

### Ethyl 6-amino-4-oxo-1,4-dihydroquinoline-3-carboxylate (5)

To a solution of 6-nitro-4-oxo-1,4-dihydroquinoline-3-carboxylate (2.0 g, 7.63 mmol) in dimethylformamide (DMF, 40 ml), was added 10% Pd/C (0.2 g). Hydrogenation was carried out under a pressure of 1 atm at 100° C. After stirring for 12 hours, removal of the catalyst and solvent gave a solid residue, which was then washed by ethyl acetate (40 ml) to afford ethyl 6-amino-4-oxo-1,4-dihydroquinoline-3-carboxylate (1.5 g, 85% yield). ¹H NMR (500 MHz, DMSO) δ 12.03 (s, ¹H), 8.32 (s, ¹H), 7.33 (d, J=8.7 Hz, ¹H), 7.26 (d, J=2.6 Hz, ¹H), 6.99 (dd, J=8.7, 2.6 Hz, ¹H), 5.45 (s, ²H), 4.18 (q, J=7.1 Hz, ²H), 1.26 (t, J=7.1 Hz, ³H). LC-MS (ESI): m/z [M+H]+ calcd. For C12H13N2O3: 233.09, found: 233.10.

### (S)-ethyl 6-(2-((((9H-fluoren-9-yl)methoxy)carbonyl)amino)propanamido)-4-oxo-1,4-dihydroquinoline-3-carboxylate (6)

Fmoc-L-Ala-OH (2.0 g, 6.42 mmol), HOBt (1.13 g, 8.35 mmol) and HBTU (3.17 g, 8.35 mmol) were dissolved in dry dimethylformamide (DMF, 40 ml). The mixture was stirred at room temperature for 15 min. Ethyl 6-amino-4-oxo-1,4-dihydroquinoline-3-carboxylate (1.34 g, 5.78 mmol) and N,N-Diisopropylethylamine (3.4 ml, 19.27 mmol) were then added and the resulting mixture was stirred at room temperature over night. DMF was removed by rotary evaporator, ethyl acetate and water were then added. The formed precipitate was collected by filtration and purified by column chromatography eluting with dichloromethane/methanol 10:1 v/v to give the Fmoc-protected intermediate (5) as a light brown solid (2.2 g, 65% yield). ¹H NMR (500 MHz, DMSO) δ 12.28 (d, J=6.4 Hz, 10.27 (s, ¹H), 8.47 (d, J=6.5 Hz, ¹H), 8.39 (d, J=2.1 Hz, ¹H), 7.95 (dd, J=8.9, 2.3 Hz, ¹H), 7.88 (d, J=7.6 Hz, ²H), 7.75-7.68 (m, ³H), 7.57 (d, J=8.9 Hz, ¹H), 7.44-7.37 (m, ²H), 7.35-7.28 (m, ²H), 4.28-4.26 (m, ²H), 4.21-4.17 (m, ⁴H), 1.32 (d, J=7.1 Hz, ³H), 1.26 (t, J=7.1 Hz, ³H). LC-MS (ESI): m/z [M+H]+ calcd. For C₃₀H₂₈N₃O₆: 526.20, found: 526.30.

### (S)-ethyl 6-(2-aminopropanamido)-4-oxo-1,4-dihydroquinoline-3-carboxylate (7)

The Fmoc-protected intermediate 6 (2.1 g, 4.0 mmol) was dissolved in DMF (30 ml). Piperidine (7.5 ml) was added and the reaction mixture stirred at room temperature for 1 hour. Concentration in vacuo gave a brown solid which was washed by ethyl acetate to afford the title compound (0.83 g, 69% yield). ¹H NMR (500 MHz, DMSO) δ 8.49 (s, 8.43 (d, J=2.4 Hz, ¹H), 7.98 (dd, J=8.9, 2.4 Hz, ¹H), 7.58 (d, J=8.9 Hz, ¹H), 4.21 (q, J=7.1 Hz, ²H), 3.52-3.47 (m, ¹H), 1.27 (t, J=7.1 Hz, 3H), 1.25 (d, J=6.9 Hz, 3H). LC-MS (ESI): m/z [M+H]+ calcd. For C₁₅H₁₈N₃O₄: 304.13, found: 304.20.

### (S)-ethyl 6-(2-([1,1′-biphenyl]-4-ylcarboxamido)propanamido)-4-oxo-1,4-dihydroquinoline-3-carboxylate (8)

Biphenyl-4-carboxylic acid (0.40 g, 2.02 mmol), HOBt (0.35 g, 2.62 mmol) and HBTU (1.0 g, 2.62 mmol) were dissolved in dry dimethylformamide (DMF, 20 ml). The mixture was stirred at room temperature for 15 min. (S)-ethyl 6-(2-aminopropanamido)-4-oxo-1,4-dihydroquinoline-3-carboxylate (0.55 g, 1.82 mmol) and N,N-Diisopropylethylamine (1.07 ml, 6.05 mmol) were then added and the resulting mixture was stirred at room temperature overnight. DMF was removed by rotary evaporator, ethyl acetate and water were then added. The formed precipitate was collected by filtration and washed by ethyl acetate to give (S)-ethyl 6-(2-([1,1′-biphenyl]-4-ylcarboxamido)propanamido)-4-oxo-1,4-dihydroquinoline-3-carboxylate (0.76 g, 78% yield). ¹H NMR (500 MHz, DMSO) δ 12.30 (d, J=6.7 Hz, ¹H), 10.36 (s, ¹H), 8.74 (d, J=7.0 Hz, ¹H), 8.48 (d, J=6.7 Hz, ¹H), 8.41 (d, J=2.3 Hz, ¹H), 8.03 (d, J=8.4 Hz, ²H), 8.00 (dd, J=8.9, 2.4 Hz, ¹H), 7.79 (d, J=8.4 Hz, ²H), 7.75 (d, J=7.2 Hz, ²H), 7.59 (d, J=8.9 Hz, ¹H), 7.50 (t, J=7.6 Hz, ²H), 7.41 (t, J=7.4 Hz, ¹H), 4.67-4.61 (m, ¹H), 4.21 (q, J=7.1 Hz, ²H), 1.48 (d, J=7.2 Hz, ³H), 1.28 (t, J=7.1 Hz, ³H). LC-MS (ESI): m/z [M+H]+ calcd. For C₂₈H₂₆N₃O₅: 484.19, found: 484.20.

### (S)-6-(2-([1,1′-biphenyl]-4-ylcarboxamido)propanamido)-4-oxo-1,4-dihydroquinoline-3-carboxylic acid (L-1)

To a solution of compound 8 (500 mg, 1.03 mmol) in Methanol (20 ml) and H₂O (20 ml), KOH (580 mg, 10.34 mmol) was added. The obtained mixture was stirred at 60° C. for 16 hours. The mixture was brought to 0° C., carefully acidified with 1N HCl until pH=1. The formed precipitate was collected by filtration and purified by HPLC to furnish the desired product L-1 as an off-white solid (422 mg, 90% yield). ¹H NMR (500 MHz, DMSO) δ 10.53 (s, ¹H), 8.83 (d, J=6.8 Hz, ¹H), 8.78 (d, J=6.8 Hz, ¹H), 8.65 (d, J=2.4 Hz, ¹H), 8.10 (dd, J=9.1, 2.4 Hz, ¹H), 8.04 (d, J=8.5 Hz, ²H), 7.84-7.77 (m, 3H), 7.76-7.70 (m, ²H), 7.50 (t, J=7.6 Hz, ²H), 7.45-7.39 (m, ¹H), 4.68-4.61 (m, ¹H), 1.49 (d, J=7.2 Hz, ³H). ¹³C NMR (126 MHz, DMSO) δ 177.99 (s), 172.03 (s), 166.55 (s), 166.08 (s), 144.05 (s), 142.89 (s), 139.15 (s), 137.34 (s), 135.40 (s), 132.67 (s), 129.04 (s), 128.30 (s), 128.08 (s), 126.88 (s), 126.41 (s), 126.19 (s), 124.95 (s), 120.41 (s), 113.21 (s), 107.07 (s), 50.13 (s), 17.61 (s). LC-MS (ESI): m/z [M−H]− calcd. For C₂₆H₂₀N₃O₅: 454.14, found: 454.30. HRMS (ESI-TOF): m/z [M−H]− calcd. For C₂₆H₂₀N₃O₅: 454.1403, found: 454.1413; Purity: >95% (UV, λ=254 nm).

Enzyme Kinetic Assay.

PTP activity was assayed using p-nitrophenyl phosphate (pNPP) as a substrate in 3,3-dimethylglutarate buffer (50 mM 3,3-dimethylglutarate, pH 7.0, 1 mM EDTA, 150 mM NaCl) at 25° C. The assays were performed in 96-well plates. Normally, to determine the IC50 values for PTPN22, the reaction was initiated by the addition of enzyme (final concentration at 20 nM) to a reaction mixture (final volume 0.2 mL) containing 5.0 mM (Km for the substrate against PTPN22) pNPP with serial dilutions. To determine the IC50 values for other PTPs, the assays were carried out under the same conditions used for PTPN22 except that the concentration of the pNPP was set at the corresponding Km value for each PTP. All PTPs used in the study were recombinant proteins prepared in-house. Concentration of compounds used to determine IC50 values ranged from 0.2- to 5-fold of the IC50 values. The reaction rate was measured using a SpectraMax Plus 384 microplate spectrophotometer (Molecular Devices). To determine the mode of inhibition, the reactions were initiated by the addition of PTPN22 (final concentration at 20 nM) to the reaction mixtures (0.2 mL) containing various concentrations of pNPP and inhibitor L-1. Data were fitted using SigmaPlot Enzyme Kinetics Module (Systat Software, Inc.).

Pharmacokinetics Study for L-1.

L-1 is a novel PTPN22 inhibitor with IC50 as low as 1.4±0.2 μM. In order to study the effects of L-1 in mouse model, pharmacokinetics data is required to understand its absorbance/distribution/metabolism/elimination (ADME) properties. The detailed experimental procedures and resulting pharmacokinetics parameters are present below.

Animal Dosing and Sample Collection for Pharmacokinetic Studies.

The compound L-1 was first dissolved in DMSO to make a 20 mg/ml solution. Then the solution was further diluted to a 2 mg/ml solution for which the formulation is 10% DMSO-85% PBS-5% Cremophor EL (CrEL). Each mouse was administered a single IP dose of 10 mg/kg. The volume of each injection was about 100 μL according to the weight of mouse. At different time points (1 h, 1.5h, 2 h, 2.5h, 3 h, 6, 24 h), blood samples (50 μL) were collected and centrifuged to get the serum. The serum (10 μL) were then mixed with acetonitrile (20 μL) and centrifuged. The supernatant were collected and subjected to Liquid Chromatography/Mass Spectrometry analysis.

Liquid Chromatography/Mass Spectrometry Analysis.

The Liquid Chromatography/Mass Spectrometry (LC/MS) analysis was carried out on a Agilent 1260 analytic HPLC system and an Agilent 6470 Triple Quadrupole MS detector, equipped with a Kinetex 2.6 um C18 column (3 mm×50 mm), eluted with 0-100% MeOH-H2O with 0.1% (w/v) formic acid at 0.7 mL/min flow-rate (gradient method: 1.2 min 0-10% MeOH linear gradient, 1.5 min 10-90% MeOH linear gradient, followed by 1.3 min 90-100% MeOH, followed by 2.5 min 100% MeOH), MS detector were set at single ion mode (SIM), monitoring the negative charge 454.2 (M−1). The detection limit for compound L-1 is 100 nM at 4 μL sample injection.

Data Analysis

The pharmacokinetic parameters were calculated in GraphPad Prism 6, and the results are shown in Table 1.

TABLE 1
The Pharmacokinetic Data for the Compound L-1.

| | | | IP | Cₘₐₓ | tₘₐₓ | | |
|---|---|---|---|---|---|---|---|
| Mouse | Conc. | formulation | dose | (μM) | (h) | kₑ | t_{1/2} (h) |
| #1 | 2 | 10% DMSO- | 10 | 1.31 | 1.5 | 0.583 | 1.189 |
| | mg/ml | 85% PBS- | mg/kg | | | | |
| | | 5% CrEL | | | | | |
| #2 | 2 | 10% DMSO- | 10 | 1.09 | 2 | 0.259 | 2.681 |
| | mg/ml | 85% PBS- | mg/kg | | | | |
| | | 5% CrEL | | | | | |
| #3 | 2 | 10% DMSO- | 10 | 1.12 | 2 | 0.285 | 2.428 |
| | mg/ml | 85% PBS- | mg/kg | | | | |
| | | 5% CrEL | | | | | |
| Average | 2 | 10% DMSO- | 10 | 1.11 | 2 | 0.341 | 2.031 |
| | mg/ml | 85% PBS- | mg/kg | | | | |
| | | 5% CrEL | | | | | |

Antibodies

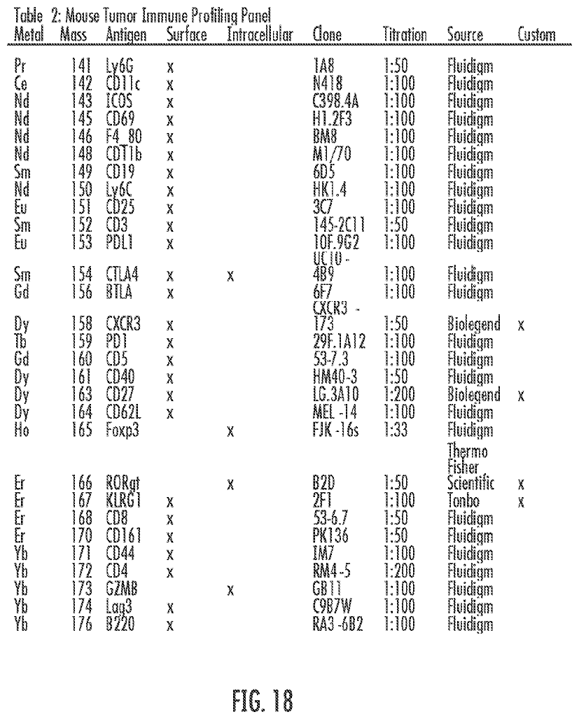

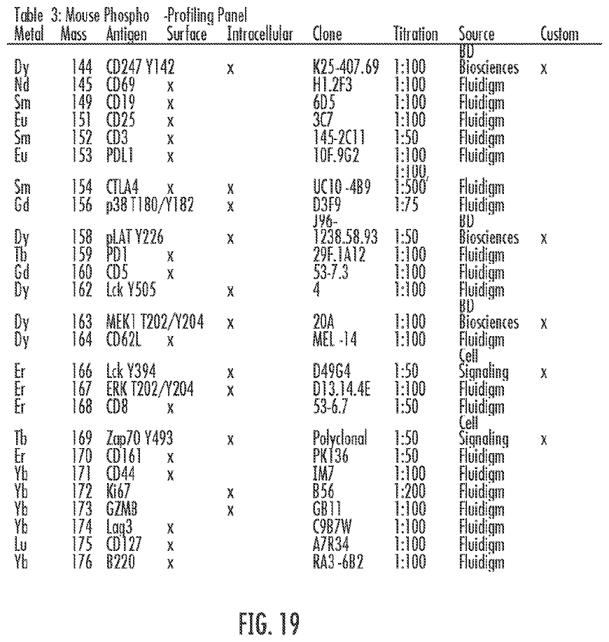

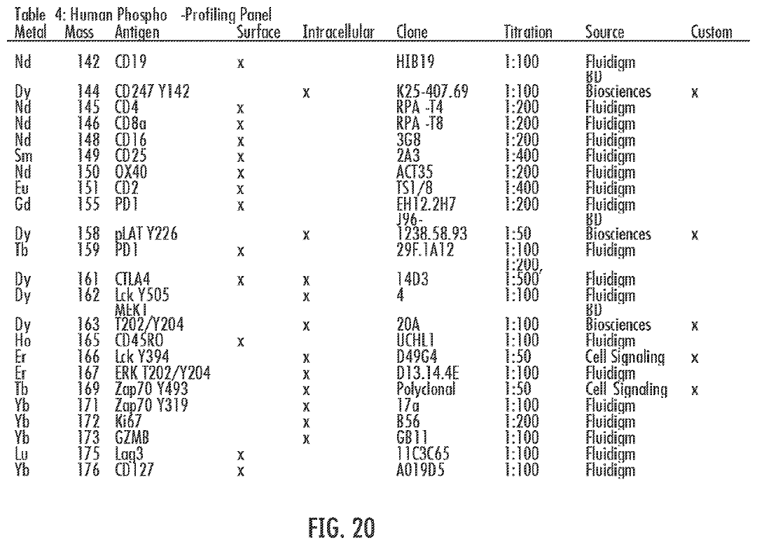

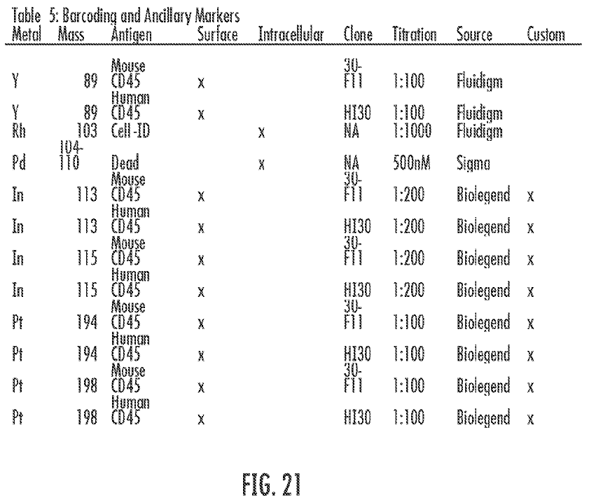

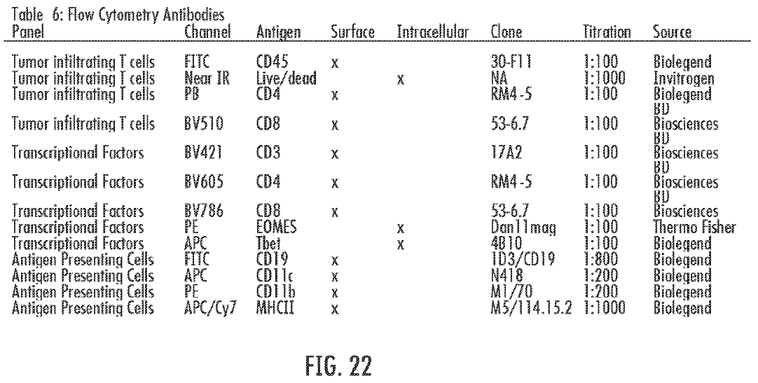

All antibodies used for CyTOF and flow cytometry experiments are listed in Tables 2-6 on FIGS. 18-22 below.

CyTOF Staining and Data Analysis

All CyTOF staining and analysis were performed as previously reported³⁷.

Briefly, 1.5×10⁶ cells from each tumor were plated in 96-well plates and washed once in PBS with 2 mM EDTA. For all immune profiling assays, cells were incubated for 5 minutes at room temperature (RT) in 500 nM 104-110Pd (Sigma) in PBS for viability staining. Barcoding was performed by incubating samples in anti-CD45 antibodies conjugated to unique metal tags³⁸. After two washes in cell staining buffer (CSB, Fluidigm), samples were multiplexed. Samples were first stained with Fc block (2 μg/1-3×10⁶ cells, BD Biosciences) for 10 minutes at RT, the surface stain cocktail for 30 minutes at RT, washed twice in CSB, and then fixed/permeabilized using Foxp3 staining kit (eBiosciences) according to the manufacturer's instructions. For phospho-profiling assays, cells plated in 96-well plates and were rested in serum-free RPMI for 2 hours at 37 C in 5% CO₂. Cells were moved to ice for incubation with 3.1 mM H₂O₂ for 5 minutes in RT or anti-CD3 (1 μg/100 μL; 145-2C11 for hamster anti-mouse; OKT3 for mouse anti-human) for 15 minutes on ice followed by cross-linking secondary antibodies (goat anti-mouse IgG 1 μg/100 μL, ThermoFisher; mouse anti-hamster IgG 0.5 μg/100 μL, BD Biosciences) for 15 minutes on ice. Cells were then incubated at 37° C. for 5 minutes and fixed immediately in 1.6% paraformaldehyde (PFA, ThermoFisher). After centrifugation, cells were washed twice with CSB, Fc blocked (as detailed above for mouse samples; Purified Human Fc Receptor Binding Inhibitor for human samples, eBioscience) for 10 minutes at RT, stained for the surface markers for 30 minutes at RT, washed again with CSB, and permeabilized with 4° C. MeOH for 10 minutes on ice. Samples were again washed twice with CSB. For either application, the fixed/permeabilized samples were stained using the intracellular cocktail stain for 30 minutes at RT. After two additional washes, cells were stored in 1.6% fresh PFA in PBS until the day of acquisition up to 7 days. On the day of data acquisition, samples were stained with 1:1000 103Rh in Maxpar Fix/Perm solution (Fluidigm) for 30 minutes at RT for cell identification. After washing the samples in CSB, samples were washed with filtered double-distilled water and resuspended in normalization beads (EQ Beads, Fluidigm).

All mass cytometry data were acquired at the University of Maryland School of Medicine Center for Innovative Biomedical Resources (CIBR) Flow Cytometry and Mass Cytometry Core Facility, Baltimore, Md. All acquired data were randomized and normalized using CyTOF software (v6.7, Fluidigm) and debarcoded by manual gating using FlowJo (v10.5, BD). Dead cells were removed by manual gating of cells doubly positive for 106Pd and 108Pd. Each processed sample was exported as individual fcs files. For clustering analysis, a modified pipeline from Nowicka et al. 39 was used in R (v3.5), employing FlowSOM17 and UMAP18 algorithms. For differential analyses, linear mixed modeling was used as indicated and FDR-adjusted p-values were obtained.

Flow Cytometry

For each sample, 1.5×10⁶ cells were plated in 96-well plates and washed with PBS containing 2 mM EDTA. Cells were Fc blocked for 10 minutes at 4° C. after which the samples were incubated in the surface staining cocktail for 30 minutes on 4° C. For tetramer analysis, cells were first stained with H2Kb SIINFEKL tetramers (NIH Tetramer Core) for 45 minutes at RT. After washing three times with cell staining buffer, cells were stained with anti-CD8 antibody (1:100, clone: KT15, MBL International) for 30 minutes at 4° C. For phospho-flow validation of clones and staining, the same stimulation and staining protocol detailed for mass cytometry was used. For transcriptional factors, cells were fixed/permeabilized using Foxp3 staining kit and stained intracellularly for 45 minutes at RT. Before data acquisition, cells were washed twice in cell staining buffer, and data was collected on CytoFLEX (Beckman).

Immunohistochemistry

All samples were formalin fixed paraffin embedded and slides were prepared according to standard histological procedures. Immunohistochemistry for CD4, CD8, and Foxp3 was performed at the Johns Hopkins Oncology Tissue Services on a Ventana Discovery Ultra autostainer (Roche Diagnostics). Briefly, slides were dewaxed and rehydrated, and epitope retrieval was performed using Ventana Ultra CC1 buffer (Roche Diagnostics) at 96° C. for 64 minutes. Primary antibody for CD4 (1:1000, clone: ab183685, Abcam), CD8 (1:125, clone: 4SM16, ThermoFisher), or Foxp3 (1:75, clone: D608R, Cell Signaling Technologies) was applied at 36° C. for 60 minutes, followed by application of rabbit anti-rat linker antibody (1:500, Vector Labs) at 36° C. for 32 minutes, anti-rabbit HQ detection system, or anti-rabbit HQ detection system with Discovery AMP Multimer (Roche Diagnostics), respectively. Chromomap DAB IHC detection kit (Roche Diagnostics) was used. Counterstaining was done with Mayer's hematoxylin. Slides were dehydrated and mounted with coverslips.

Statistical Analysis

For differential analysis of the CyTOF datasets, linear mixed modeling was used. Unless otherwise noted, differences between two groups were tested using unpaired two-tailed t-tests using GraphPad Prism 8. Statistical analysis for significant differences in the tumor growth curves were conducted using mixed effects modeling via TumGrowth software⁴⁰. Univariate survival analyses were performed with log-rank tests using a published software³⁶.

### Example 1

PTPN22 is Associated with Immune Regulation of Cancers.

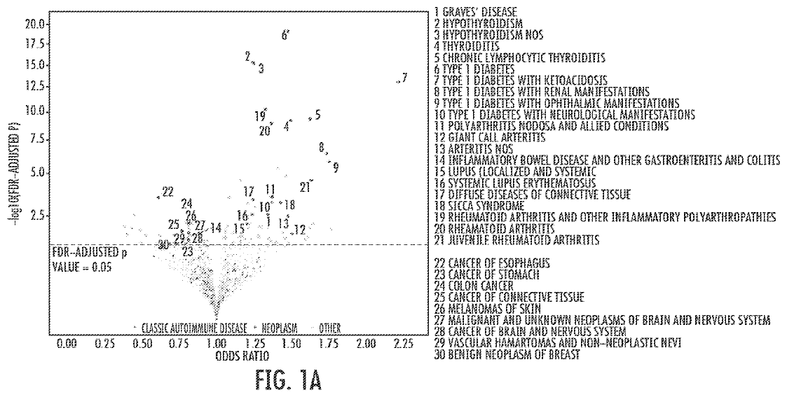

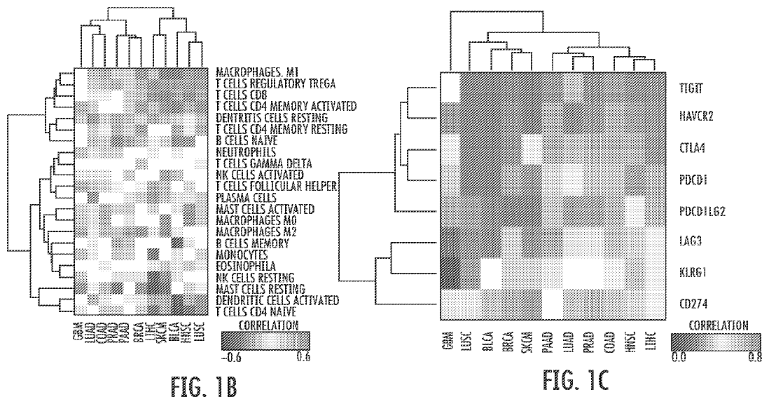

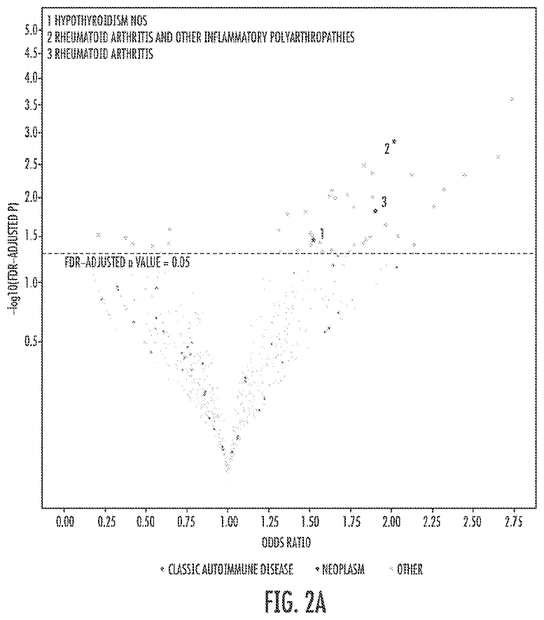

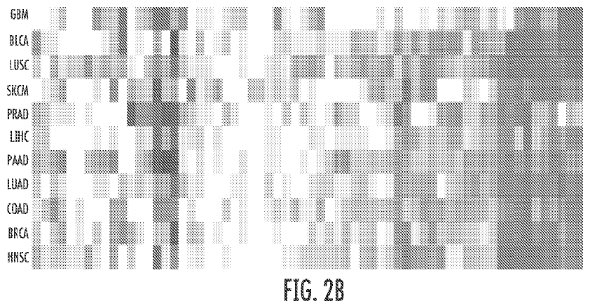

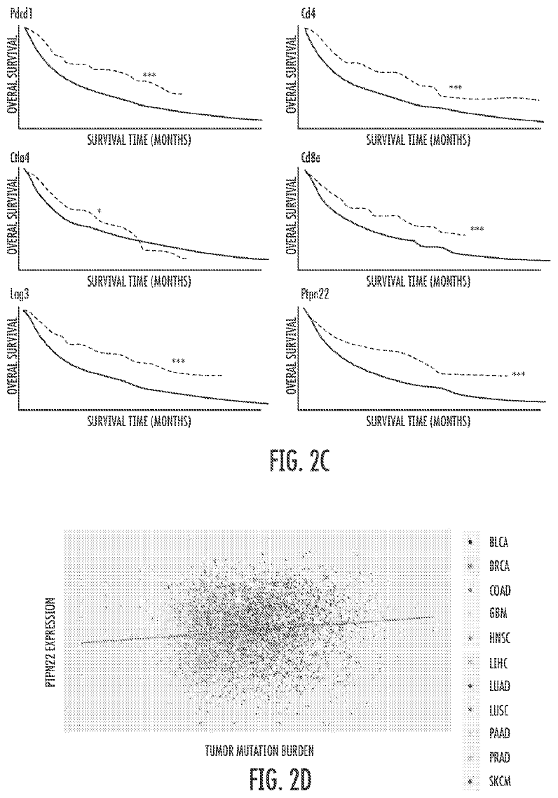

To first examine the potential role of PTPN22 in cancer development, a large PheWAS database employed that explores relationships between the germline variant PTPN22 rs2476601 and human disease phenotypes¹³. Based on genotyped DNA biobanks and de-identified electronic health records from Vanderbilt University Medical Center's BioVU, we found that the minor allele frequencies for rs2476601 were approximately 10% and 1.6% in the European (n=72,083) and African (n=14,414) ancestry populations, respectively. In the European ancestry population, we were able to recapitulate the previously reported risk-conferring associations between PTPN22 rs2476601 and classic autoimmune diseases, namely rheumatoid arthritis, systemic lupus erythematosus and thyroid disorders (FIG. 1a). Importantly, a striking risk-preventive association between PTPN22 rs2476601 and multiple types of cancers was observed, mostly ranging 0.6-0.8 odds ratios for melanoma, gastrointestinal cancers, and central nervous system cancers. In the African ancestry population, the associations were less significant (FIG. 2a).

To gain additional insight into the functional relevance of PTPN22 expression in cancers, we used the Cancer Genome Atlas database to assess the correlations between PTPN22 expression and cell type signatures across 11 cancer types. Using xCell¹⁴, a gene-set based deconvolution algorithm providing scores for over 65 cell types, we found that across seven major cancer types examined PTPN22 expression strongly correlated with scores for immune cell types and weakly correlated with scores for most other cell types (FIG. 2b). We then used a leukocyte-focused deconvolution algorithm based on linear support vector regression, CIBERSORT¹⁵, to correlate immune cell subsets with PTPN22 expression. This analysis revealed that PTPN22 expression most highly correlated with T cell and inflammatory (M1) macrophage subsets, especially in melanoma (FIG. 1b). In inflamed cancer types, PTPN22 expression also correlated strongly with the expression of other markers of immune regulation (Pdl1, Pdcd1, Ctla4, Lag3, Tim3) (FIG. 1c). When stratifying by level of expression (top 20% vs. rest), univariate survival analysis results were similar to other key immune markers, e.g. Cd4, Cd8a, Pdcd1, and Ctla4 (FIG. 2c). Expression of PTPN22 was weakly correlated (0.142) with overall tumor mutational burden (FIG. 2d). Together, these results suggested that PTPN22 is involved in the negative regulation of anticancer immunity.

### Example 2

Lack of PTPN22 Augments Anticancer Immunity.

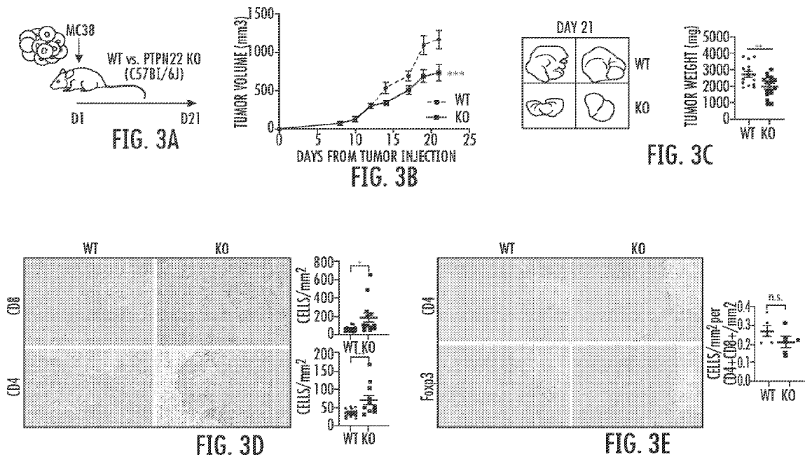

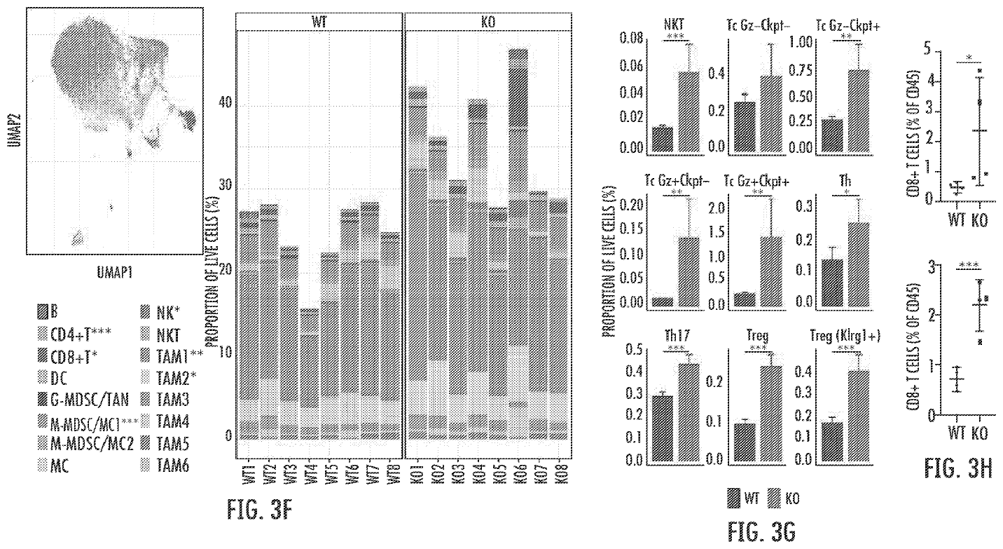

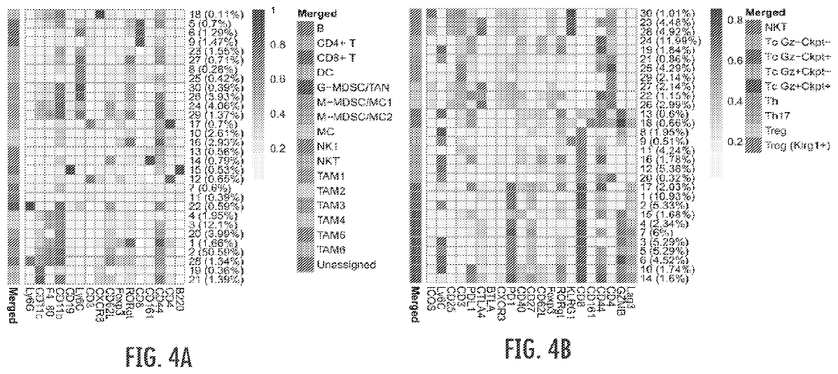

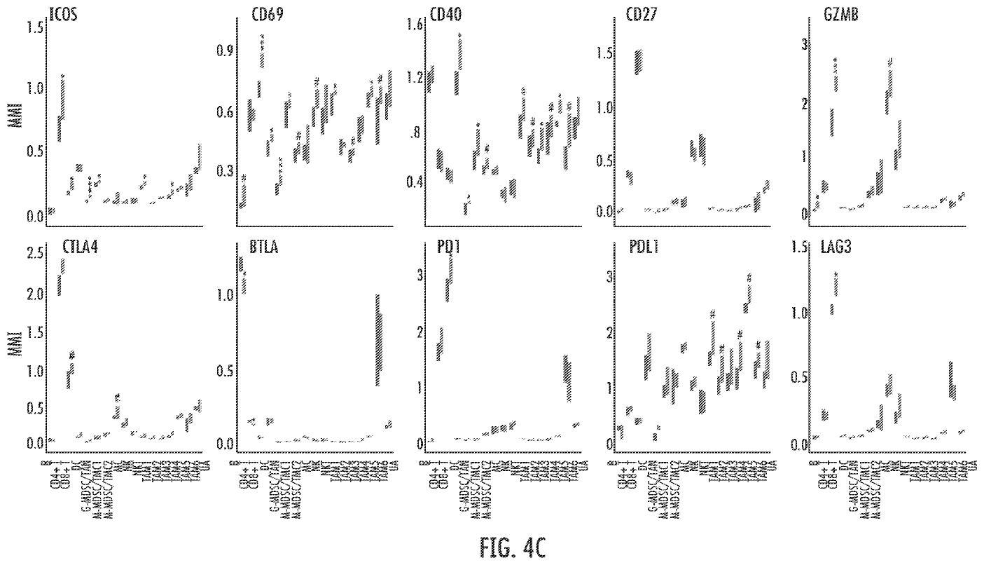

Building on these observations, the inventors directly interrogated the role of PTPN22 in anticancer immune responses by using PTPN22 knockout (KO) mice¹¹. First, MC38 carcinoma cells in C57Bl/6 background were subcutaneously injected into wild-type (WT) or PTPN22 KO C57Bl/6 mice to compare tumor growths in a syngeneic immunocompetent setting (FIG. 3a). As expected, tumor growth, gross tumor sizes, and tumor weights were significantly lower in PTPN22 KO mice compared to WT (FIG. 3b, c). Characterizing the tumors by immunohistochemistry demonstrated significantly increased presence of CD4+ and CD8+ T cells within the tumors without relative increases in Foxp3+ cells (FIG. 3d, e). Global immune-profiling of the tumors was performed using Cytometry by Time-of-Flight (CyTOF)¹⁶ analyzed by FlowSOM¹⁷ and UMAP¹⁸ algorithms, identifying a total of 16 immune cell subtypes (FIG. 4a). Many cell types, including T cells, myeloid subsets, macrophage subsets, dendritic cells, and NK cells were increased in the PTPN22 KO tumors (FIG. 3e). A more focused analysis of the same CyTOF dataset on 9 subtypes within the T cell population (FIG. 3g, FIG. 4b), showed that among the subtypes that are increased, cytotoxic CD8+ T cells expressing granzyme B were increased to the greatest extent in the tumors from PTPN22 KO vs. WT mice. Significant increases in both CD4+ and CD8+ T cells within the tumors were further confirmed by flow cytometry (FIG. 3h). In addition, functional marker profile analysis showed higher expression of CD40, CD69, and PD-L1 in the myeloid subsets and CD69 and granzyme B in the NK cells in PTPN22 KO, indicating enhanced activation in these cell types as well (FIG. 4c, FIG. 30.

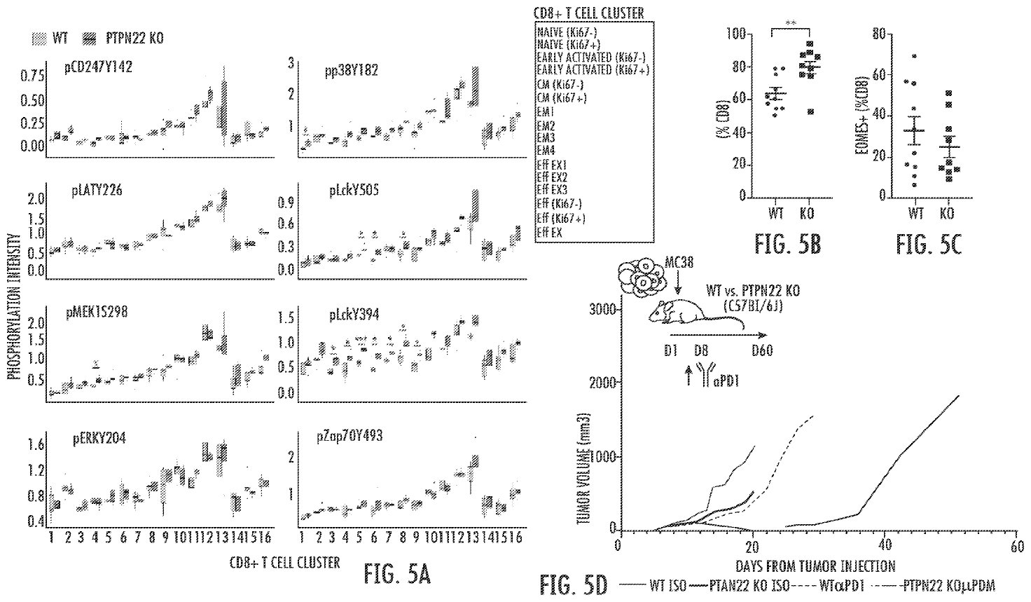

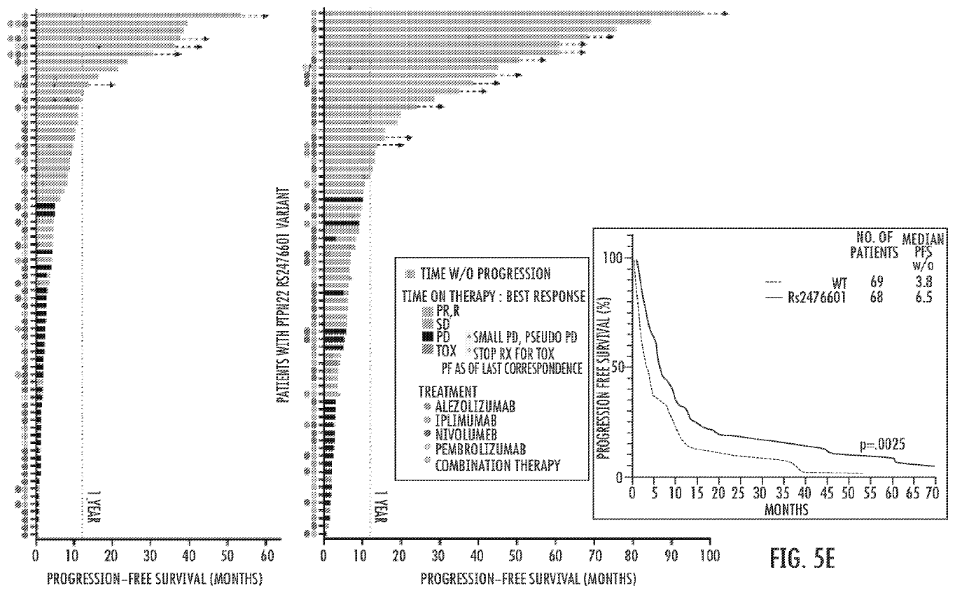

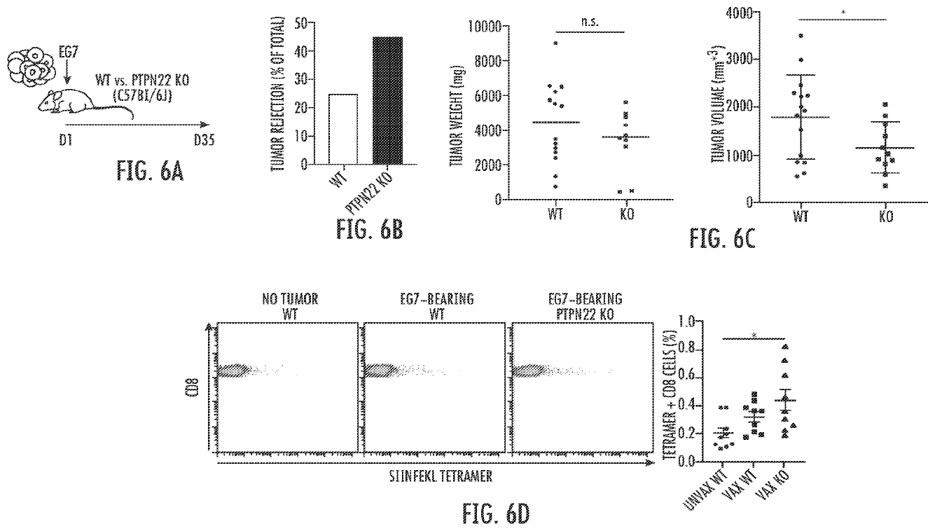

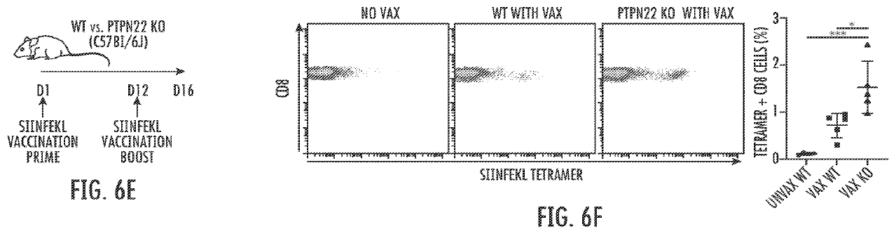

To corroborate the antitumor effects observed in mice lacking PTPN22, we tested a second tumor model, the lymphoma cell line EL4-OVA injected subcutaneously into syngeneic C57Bl/6J mice (FIG. 5a). Since EL4-OVA tumor growth appeared to be more variable, the rate of rejection was assessed instead. By day 35 from the day of tumor injection, PTPN22 KO mice had nearly double the rate of tumor rejection compared to WT mice (9/20 vs. 5/20) and smaller non-rejected tumors (FIGS. 5b-c). Additionally, the OVA-secreting nature of the tumor was exploited to determine whether the antitumor immune response is truly tumor-specific. Tetramer analysis demonstrated that PTPN22 KO mice had the highest number of SIINFEKL-specific tetramer positive CD8+ T cells within the tumor draining lymph nodes (FIG. 5d). Since day 35 from the point of tumor injection, i.e. initial antigen exposure, is a late time point and may not be sensitive enough to detect substantial differences, we compared the degree of antigen-specific responses seen in the PTPN22 KO vs. WT mice using a peptide vaccination strategy instead (FIG. 5e). Indeed, when WT and PTPN22 KO mice were vaccinated with SIINFEKL peptides, PTPN22 KO mice generated significantly greater numbers antigen-specific CD8+ T cells (FIG. 5f).

### Example 3

Phosphorylation of PTPN22-Specific Phospho-Sites Indicates CD8+ T Cell Activation.

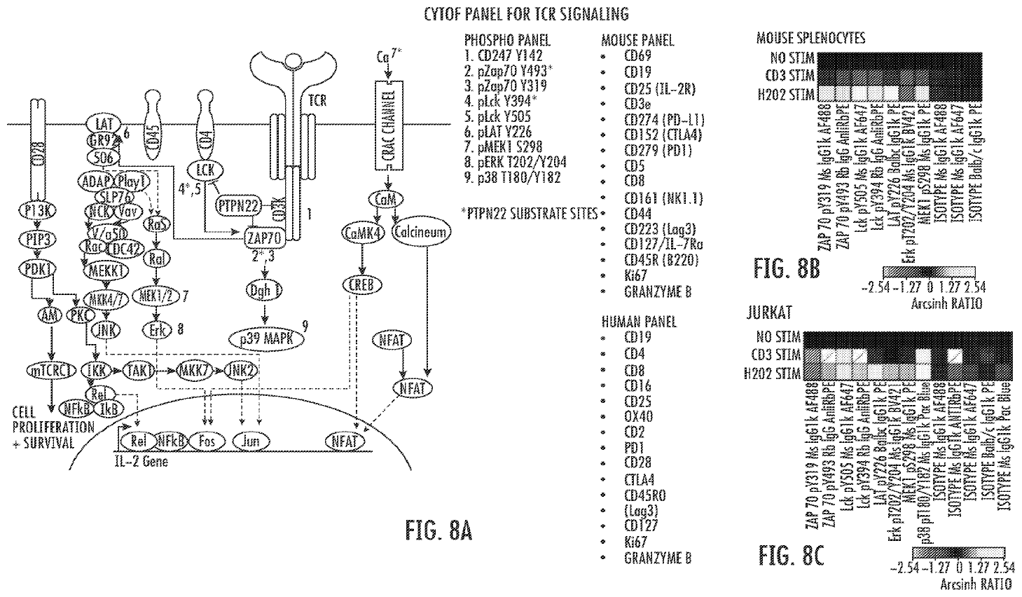

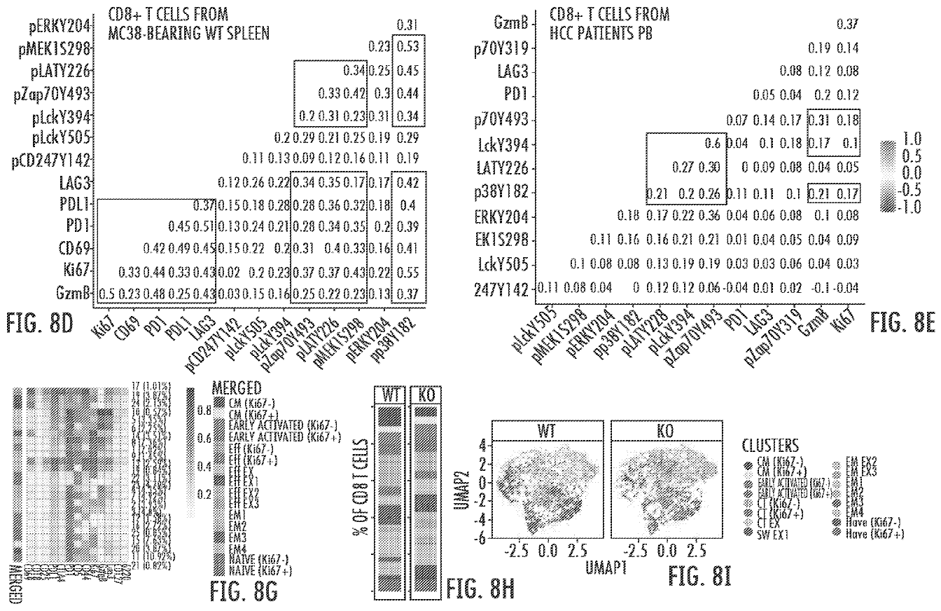

Since the infiltration of granzyme B+CD8+ T cells into the tumors was most prominently enhanced in the PTPN22 KO mice, and PTPN22 is responsible for removing activating tyrosine phosphorylation sites in Lck and Zap70, it was hypothesized that the phosphorylation states of PTPN22-specific tyrosine residues, Lck Y394 and/or Zap70 Y493, may correlate with markers of activation within CD8+ T cells. To assay the states of key phosphorylation sites along the TCR signaling cascade simultaneously with multiple lineage and functional markers at the single cell level, a CyTOF panel for phospho-immune profiling of CD8+ T cells was developed and validated (FIGS. 8a-c). Using this CyTOF panel, we first phospho-profiled CD8+ T cells from MC38 tumor-bearing mouse spleens. We chose to look at the spleen to explore correlations among the markers assayed since there would be wider dynamic ranges. In the resulting correlation matrix (FIG. 8d), we first observed that among the TCR signaling phospho-sites, Zap70 Y493, Lck Y394, LAT Y226, MEK1 5298 and p38 MAPK Y182 were highly correlated; CD247 Y142, Lck Y505 and ERK Y204 were less correlated. Among the functional markers, strongest correlations were noted between granzyme B and Ki67, and among the checkpoint markers. Out of all phospho-sites, Zap70 Y493, LAT 226, MEK1 S298 and p38 MAPK Y182 correlated most strongly with the activation markers. We then sought to verify these correlations using an independent set of samples. Since many of the antibody clones were generated specifically to human epitopes, we phospho-profiled CD8+ T cells to obtain a comparable correlation matrix using de-identified peripheral blood samples from patients with hepatocellular carcinoma naive to immunotherapy (Data FIG. 8e). Correlations were generally weaker in the human dataset, but the most appreciable correlation was among Zap70 Y493, Lck Y394, LAT Y226 and p38 MAPK Y182, similar to what was observed in mice. The highest correlation between phospho-sites was seen between the two PTPN22-specific sites Zap70 Y493 and Lck Y394. Among the functional markers, correlation between granzyme B and Ki67 was again the strongest. Also consistent with the mouse dataset, Zap70 Y493, Lck Y394 and p38 MAPK Y182 highly correlated with granzyme B and Ki67 among all phospho-sites. These findings together suggested that activation of tumor-killing CD8+ T cells correlated with activating phosphorylation of PTPN22-specific sites and downstream activation of both the classical pathway, i.e. Zap7O-LAT-MEK1, and the alternative pathway^{19,20}, i.e. Zap70-p38 MAPK for TCR signal transduction.

### Example 4

Enhanced TCR Activation in PTPN22 KO CD8+ T Cells Toward Exhaustion Further Cooperates with PD1 Inhibition.

Next, to understand (i) whether the changes in the TCR signaling phospho-profiles in the CD8+ T cells within the tumor microenvironment are in fact different between WT and PTPN22 KO, and (ii) whether these changes are specific to a particular functional state of CD8+ T cells, we phospho-profiled tumor-infiltrating leukocytes from MC38 tumors using CyTOF. We clustered the dataset into 16 differentiation states of CD8+ T cells (FIGS. 8g-i) and discovered that the phosphorylation levels in all of the assayed TCR signaling phospho-sites increased in coordination with memory and “exhausted” (the expression of checkpoint markers) phenotypes (FIG. 5a). When comparing between WT and PTPN22 KO, the most significantly different phospho-site among all phospho-sites was the PTPN22-specific Lck Y394. Higher levels of phosphorylation in PTPN22 KO were noted early in the CD8+ T cell programming, being detected in early activated and central memory subtypes. These results suggested that the lack of PTPN22, i.e. thus increased activity of Lck, leads to enhanced TCR signal transduction and T cell activation, which may in turn foster augmented effector functions and differentiation toward memory and exhaustion states upon antigen recognition. This interpretation was consistent with the significantly increased infiltration of granzyme B+ T cells and checkpoint marker-expressing T cells in the MC38 tumors from PTPN22 KO mice (FIG. 3g).

The state of T cell exhaustion is a nuanced characterization. Expression of checkpoint markers occurs downstream of T cell activation and do not necessarily indicate terminal exhaustion²¹. To determine whether the lack of PTPN22 promotes terminal exhaustion, we assayed eomesodermin (EOMES) and Tbet transcriptional factors in the tumor-infiltrating CD8+ T cells by flow cytometry which can further characterize the state of exhaustion²². Interestingly, while the proportion of Tbethi CD8+ T cells was higher in the MC38 tumors from PTPN22 KO than WT (FIG. 5b), the proportion of EOMES+CD8+ T cells was not significantly different (FIG. 5c). Based on this result, we hypothesized that checkpoint inhibition would reinvigorate the Tbethi CD8+ T cells23 to further enhance the tumor-resistant phenotype of PTPN22 KO mice. Indeed, when the mice were treated with anti-PD1 therapy, the tumor-resistant effects seen in the PTPN22 KO was augmented significantly (FIG. 5d). Remarkably, a significantly enhancing effect of checkpoint immunotherapy was seen in patients with rs2476601 when compared against matched control patients, without significant differences in immune-related adverse events (Tables 7-8, FIG. 5e).

TABLE 7
Patient Characteristics

| | | rs2478601 | Control |
|---|---|---|---|
| | All Patients | SNP | Group |
| Characteristic | (N = 137) | (N = 68) | (N = 69) |
| Age at treatment-yr | | | |
| Mean ± SD | 62.6 ± 11.0 | 62. ± 12,1 | 63.5 ± 9.1 |
| Median (range) | 64 (31-86) | 63 (31-86) | 65 (33-80) |
| Sex-no (%) | | | |
| Female | 47 (34) | 31 (46) | 16 (23) |
| Male | 90 (66) | 37 (54) | 53 (77) |
| Race or ethnic group- | | | |
| no (%) | | | |
| White | 131 (96) | 67 (99) | 64 (93) |
| Black | 3 (2) | 0 | 3 (4) |
| Unknown | 3 (2) | 1 (1) | 2 (3) |
| Primary diagnosis- | | | |
| no. (%) | | | |
| HNSCC | 9 (7) | 4 (7) | 5 (7) |
| Melanoma | 42 (31) | 21 (31) | 21 (31) |
| NSCLC | 37 (27) | 19 (28) | 18 (26) |
| SCLC | 9 (7) | 3 (4) | 6 (9) |
| RCC | 17 (12) | 7 (10) | 10 (14) |
| Urothelial | 8 (6) | 6 (9) | 2 (3) |
| Other | 15 (11) | 8* (16) | 7 (10) |

*4 of the 8 Patients categorized as ″other″ contained tw o primaries. All 4 patients had lung as atleast one of their primaries. 2 of the 4 patients contained tw o lung primaries

TABLE 8
Adverse Events

| | All | r52476601 | Control |
|---|---|---|---|
| | Patients | SNP | Group |
| Event | (N = 137) | (N = 68) | (N = 69) |
| Patients with ≥ 1 | 101 (75) | 52 (77) | 49 (71) |
| adverse events noted | | | |
| during treatment-no (%) | | | |
| Patients ≥ 2 adverse events ntoed | 66 (48) | 33 (49) | 30 (44) |
| during treatment-no (%) | | | |
| Most common adverse events | | | |
| noted during treatment-no | | | |
| Colitis | 10 | 6 | 4 |
| Diarrhea | 5 | 3 | 2 |
| Dyspena | 11 | 7 | 4 |
| Electrolyte Imbalance | 6 | 1 | 5 |
| (Hypo-calcemia, | | | |
| -kalemia, -natremia) | | | |
| Fatigue | 22 | 11 | 11 |
| Fever | 6 | 3 | 3 |
| Hepatitis | 6 | 2 | 4 |
| Hypophysitis | 8 | 5 | 3 |
| Hypothyroidism | 10 | 6 | 4 |
| Pain | 8 | 5 | 3 |
| Pneumonitis | 7 | 3 | 4 |
| Rash | 10 | 5 | 5 |
| Weight loss | 6 | 5 | 1 |
| Adverse event resulting | 30 (22) | 18 (26) | 12 (17) |
| in discontinuation | | | |
| of treatment-no (%) | | | |
| Colitis | 9* | 6* | 3 |
| COPD | 2 | 2 | 0 |
| Diabetes mellitus | 1 | 1 | 0 |
| Dyspena | 1 | 1 | 0 |
| Gastritis | 1 | 1 | 0 |
| Hepatitis | 4 | 2 | 2 |
| Hypophisitis | 2* | 1 | 1* |
| Hypopituitarism | 1 | 0 | 1 |
| Limbic encephalitis | 1 | 1 | 0 |
| Mucositis | 1 | 1 | 0 |
| Neutropenia | 1 | 1 | 0 |
| Pneumonitis | 4* | 1 | 3* |
| Rash | 1 | 0 | 1 |
| Tachycardia | 1 | 0 | 1 |

*1 case in which therapy was resumed after AE w as under control

### Example 5

PTPN22 is a Systemically Druggable Immunotherapy Target.

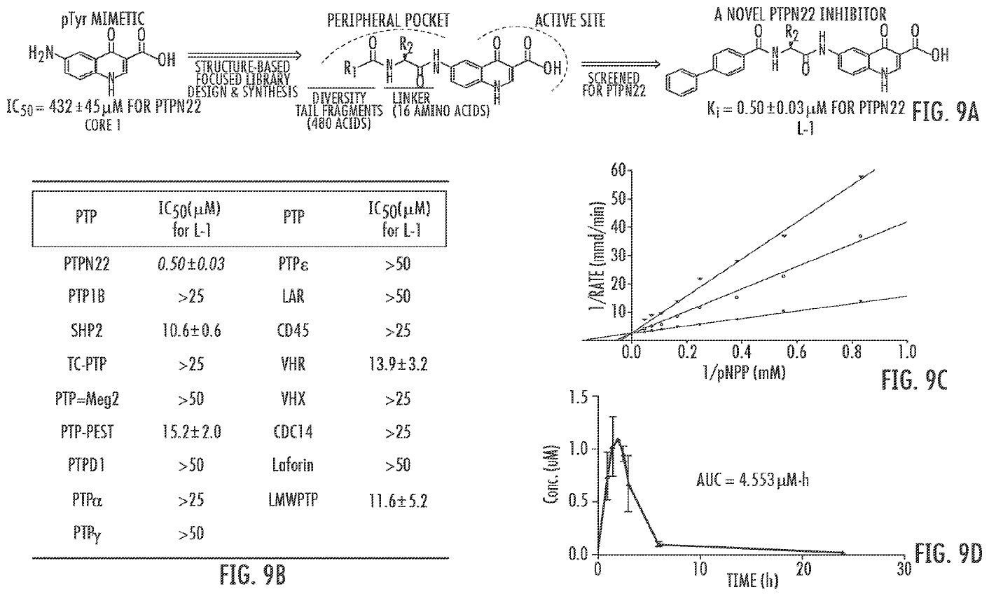

To extend the translatability of these findings, we sought to test whether systemic inhibition of PTPN22 with a pharmacological agent leads to a phenotype comparable to the genetic knockout. To identify PTPN22 inhibitors with the requisite pharmacological properties, we screened a small collection of drug-like molecules for PTPN22 inhibitory activity. We discovered a quinolone derivative (Core 1, FIG. 9a) that inhibits PTPN22 with an IC50 of 432±45 μM. Through a structure-guided and fragment-based focused library approach, compound L-1 consisting of Core 1, L-alanine as a linker, and biphenyl carboxylic group as the tail fragment, was identified as the most potent inhibitor of PTPN22 with an IC₅₀ value of 1.4±0.2 μM, which represented nearly a 310-fold gain in binding affinity compared to the parent Core 1. Selectivity profiling revealed that L-1 exhibits >7-10 fold selectivity for PTPN22 over similar phosphatases (FIG. 9b). Further kinetic analyses revealed that L-1 is a competitive inhibitor for PTPN22 with a Ki value of 0.50±0.03 μM (FIG. 9c). Since L-1 is fairly hydrophobic, we also developed a formulation utilizing an emulsifier, cremophor-EL, to improve its in vivo pharmacokinetics. Administration of L-1 intraperitoneally at 10 mg/kg yielded an average AUC of 4.55 μM·h and Cmax of 1.11 μM (FIG. 9d), which is more than twice of its Ki value.

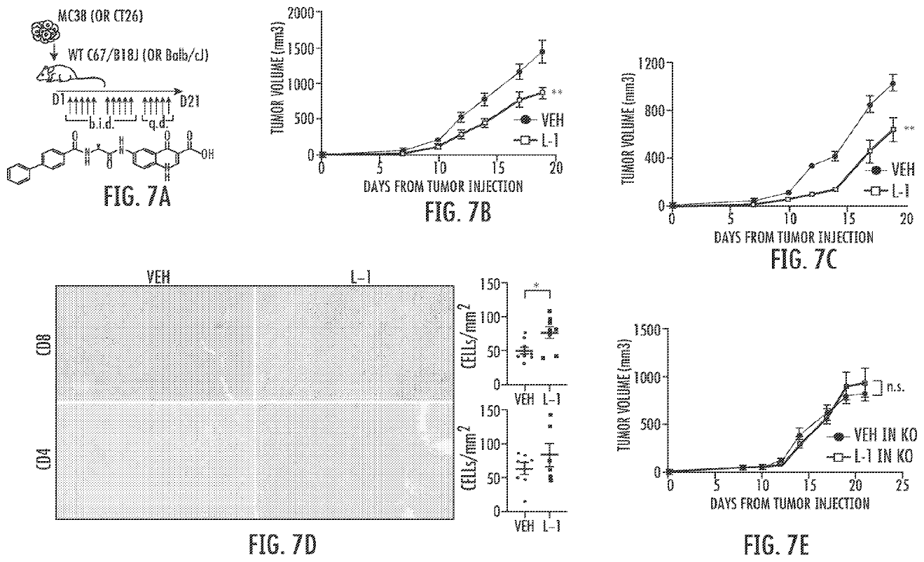

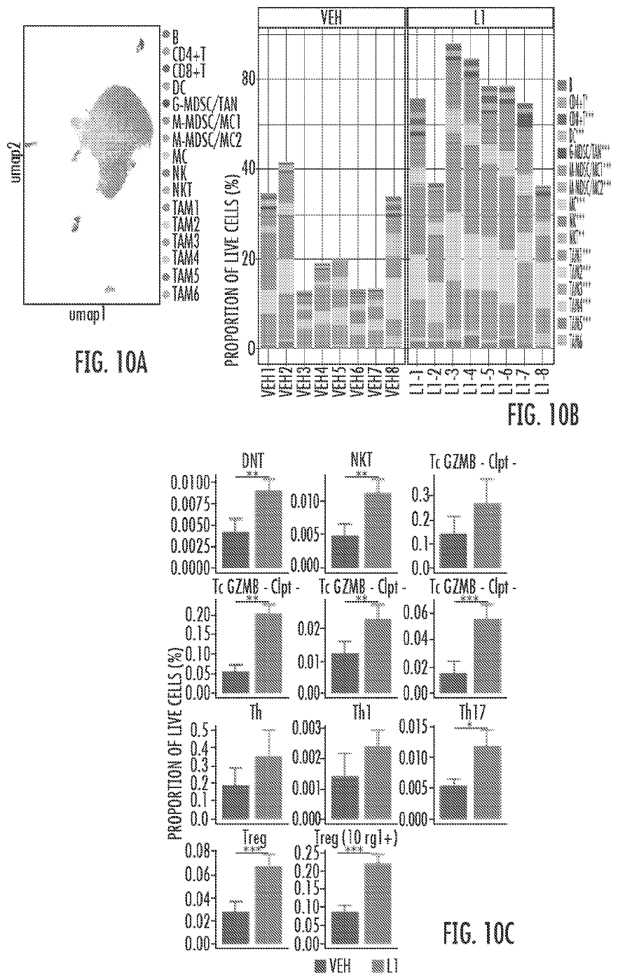

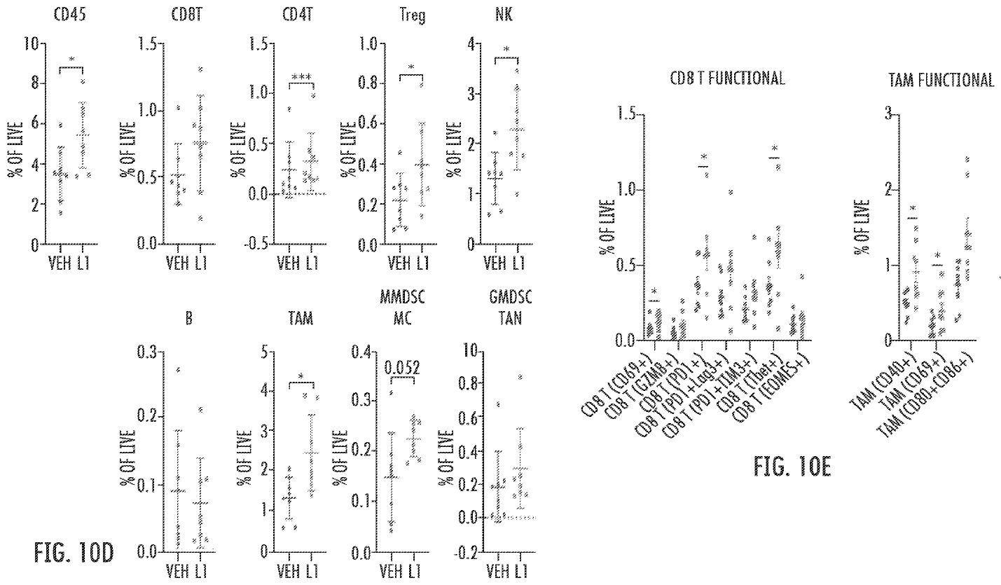

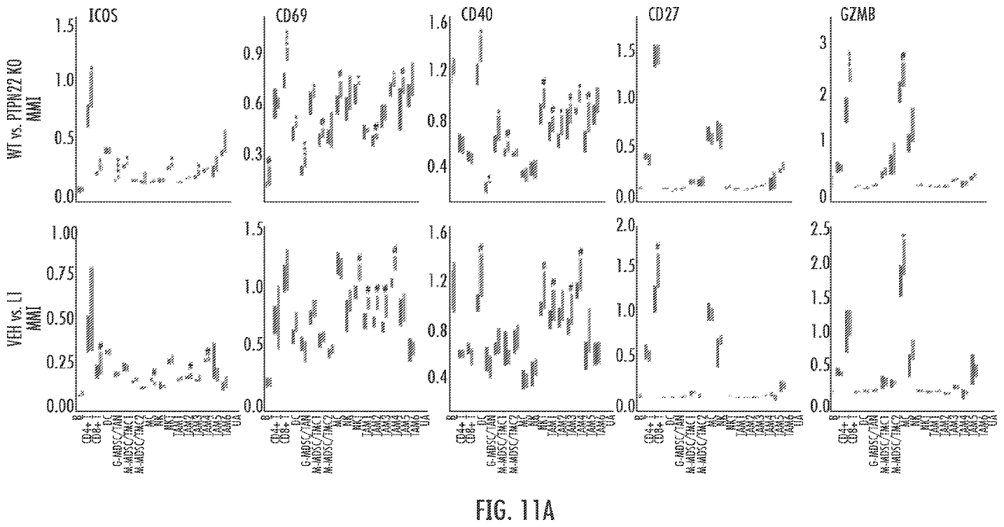

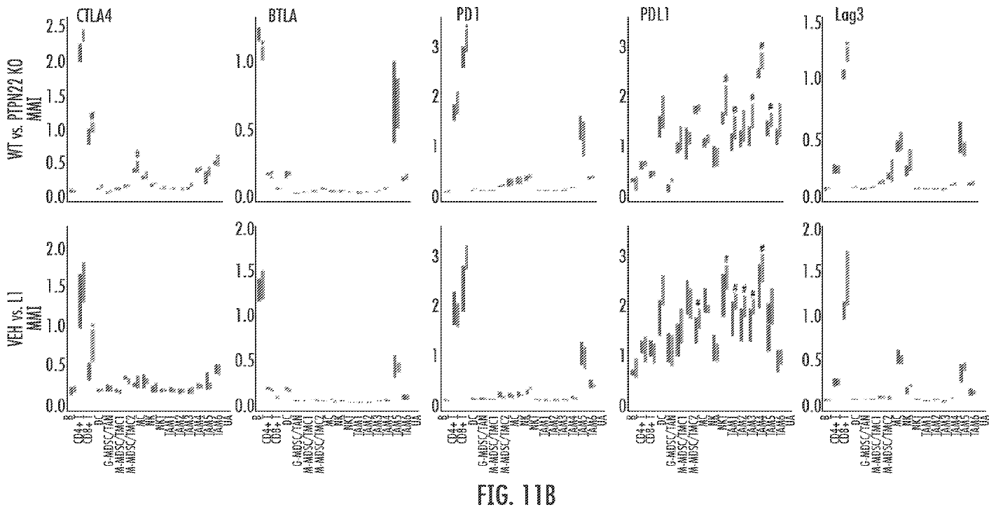

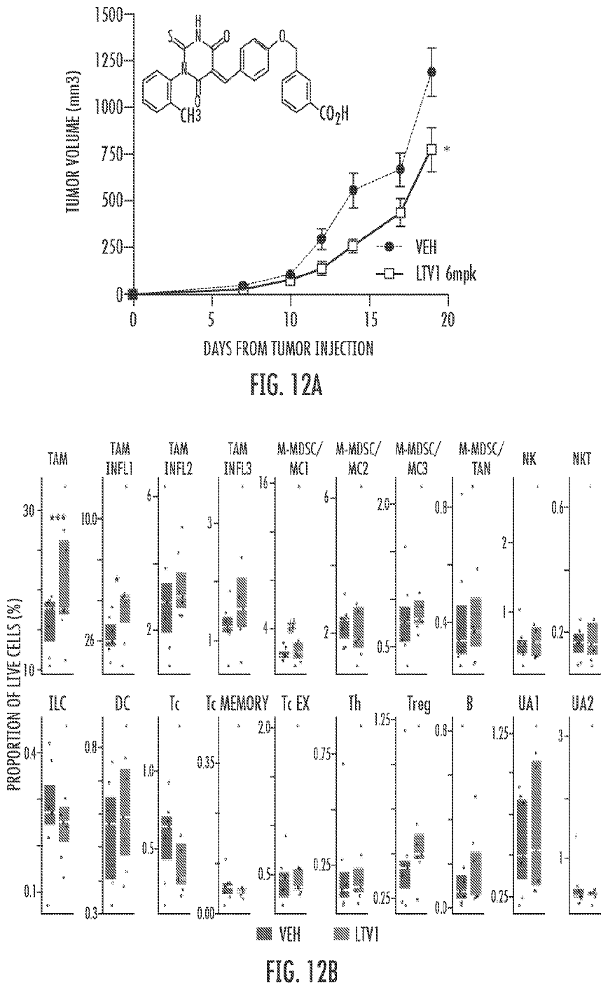

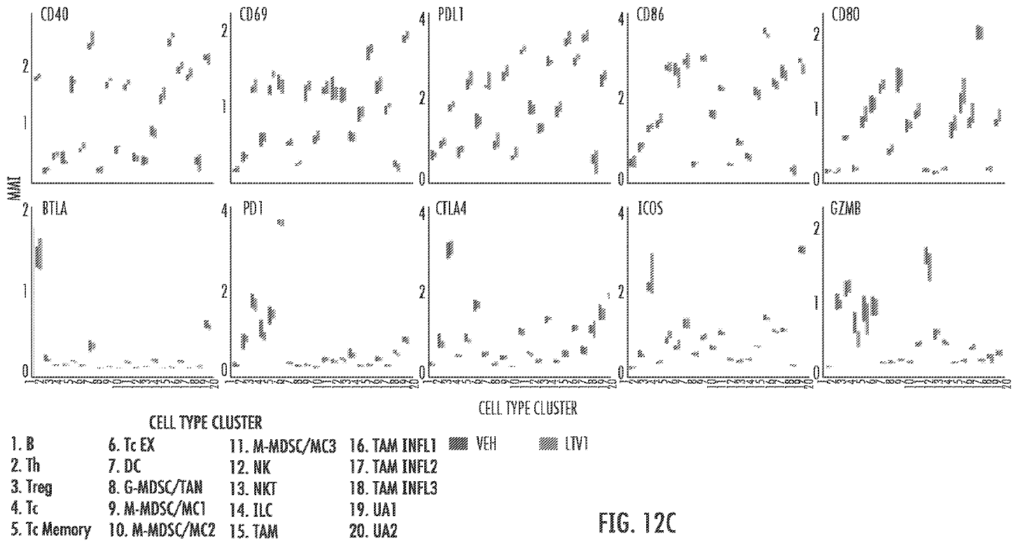

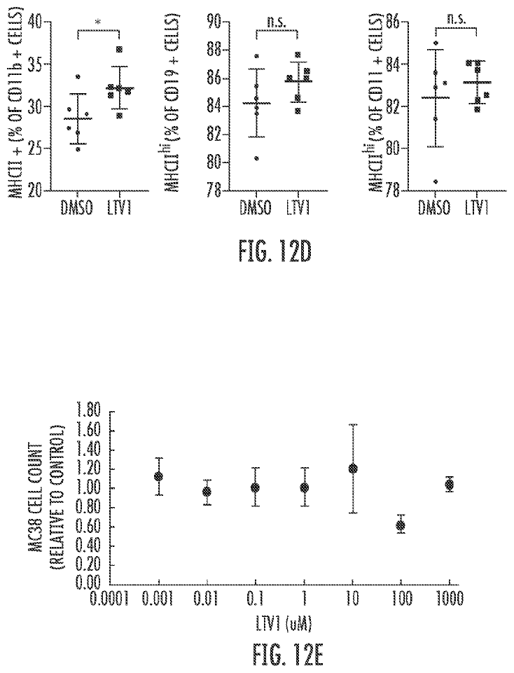

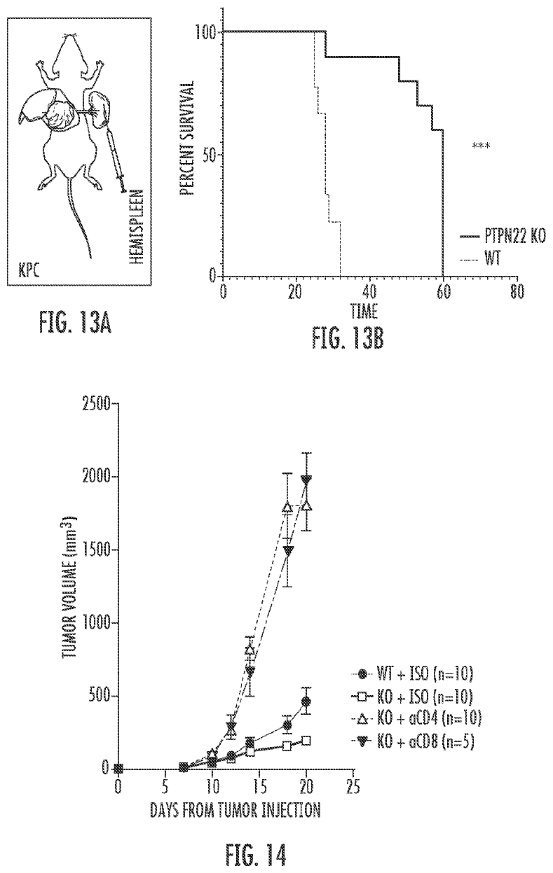

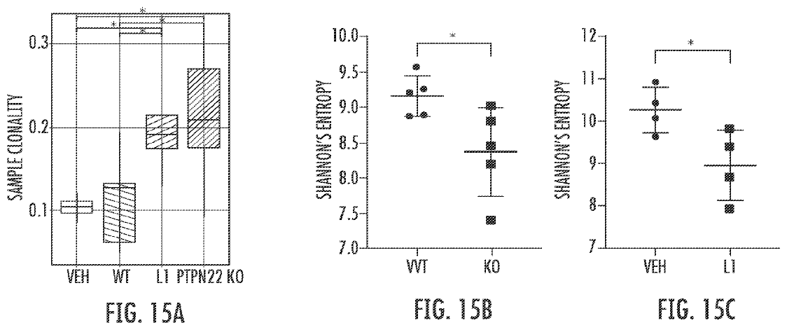

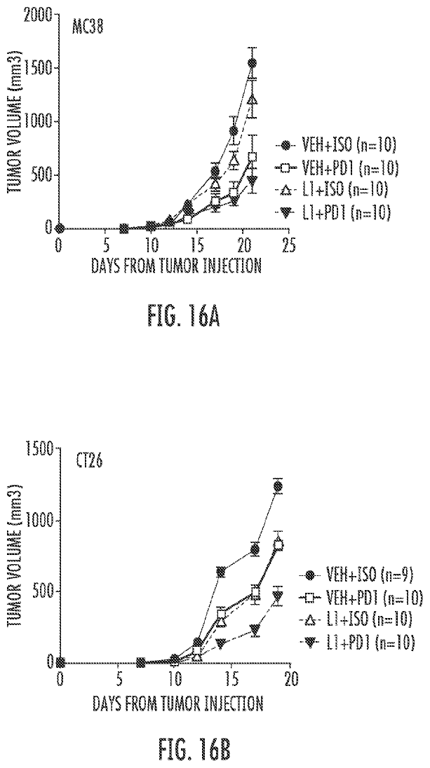

Similar to what was observed in the PTPN22 KO model, treatment of WT mice with L-1 (FIG. 7a) led to significantly reduced MC38 tumor growth compared to the vehicle-injected control group (FIG. 7b). We further tested the effect of L-1 on another syngeneic immunocompetent model, CT26 in Balb/c mice, which showed similar antitumor effects (FIG. 7c). Analysis of the MC38 tumors with immunohistochemistry demonstrated that L-1 induced increased infiltration of CD4+ and CD8+ T cells (FIG. 7d). Moreover, profiling both MC38 and CT26 tumor immune infiltrates showed significantly improved presence of multiple immune cell types and T cell subtypes in L-1 treated tumors (FIG. 10). Functional marker profiles within each of the immune cell subtype also were very comparable between the PTPN22 KO and L-1-treated conditions (FIG. 11). To test whether the effects of L-1 treatment could be attributable to off-target effects, we treated MC38 tumors in PTPN22 KO mice with either vehicle or L-1 injections. No significant differences in tumor growth were noted, suggesting that the L-1-mediated protective effects against tumor growth were off-tumor and PTPN22-specific (FIG. 7e). Subsequently, we also tested a second small molecule inhibitor of PTPN22, LTV1^{24,25}, which led to moderately decreased tumor growth (FIG. 12). Collectively, these findings showed that PTPN22 is a druggable target for immunotherapy.

The present inventions include the following: (i) PTPN22 plays a negative regulatory role in anticancer immunity; and (ii) small molecule inhibition of PTPN22 is a compelling approach to improving cancer immunotherapy. PTPN22 was previously shown to have T cell regulatory function. Consistent with our findings, adoptive cell therapy studies demonstrated that CD8+ T cells lacking PTPN22 are superior in clearing established tumors^{26,27}. The role of PTPN22 in innate immune cells have remained less clear, but there is evidence that lack of PTPN22 promotes polarization of macrophages toward inflammatory M1 phenotype²⁸, increases monocytic secretion of pro-inflammatory cytokines²⁹, and increases CD40 expression in dendritic cells (leading to higher proliferation of co-cultured CD4+ T cells)³⁰. These prior results are consistent with our own observations.

Phosphatases have historically been considered poor targets for drug development given the close similarity of the phosphatase catalytic domains. However, accumulating evidence has increasingly challenged the notion that phosphatases are not viable drug targets³¹⁻³³. Our results provide a proof-of-concept that PTPN22 can be targeted both specifically and with biological activity using a small molecule inhibitor. We anticipate that further development of small molecule inhibitors of PTPN22 will provide a novel class of immunotherapies and excite new combination strategies to be explored. Furthermore, with regards to the relatively common rs2476601 variant of PTPN22, our findings suggest that exploring the utility of the variant as a predictive or prognostic biomarker in oncology, i.e. investigating whether patients who possess the variant would require fewer cancer screening procedures or respond differently to cancer immunotherapy, would be worthwhile.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Bottini, N. & Peterson, E. J. Tyrosine Phosphatase PTPN22: Multifunctional Regulator of Immune Signaling, Development, and Disease. Annu. Rev. Immunol. 32, 83-119 (2014).
2. Stanford, S. M. & Bottini, N. PTPN22: The archetypal non-HLA autoimmunity gene. Nature Reviews Rheumatology vol. 10 602-611 (2014).
3. Rosenberg, S. A. Entering the mainstream of cancer treatment. Nat. Rev. Clin. Oncol. 11, 630-632 (2014).
4. Ribas, A. & Wolchok, J. D. Cancer immunotherapy using checkpoint blockade. Science vol. 359 1350-1355 (2018).
5. Fesnak, A. D., June, C. H. & Levine, B. L. Engineered T cells: The promise and challenges of cancer immunotherapy. Nature Reviews Cancer vol. 16 566-581 (2016).
6. Pardoll, D. M. The blockade of immune checkpoints in cancer immunotherapy. Nature Reviews Cancer vol. 12 252-264 (2012).
7. Wu, J. et al. Identification of substrates of human protein-tyrosine phosphatase PTPN22. J. Biol. Chem. 281, 11002-11010 (2006).
8. Bottini, N. et al. A functional variant of lymphoid tyrosine phosphatase is associated with type I diabetes. Nat. Genet. 36, 337-338 (2004).
9. Begovich, A. B. et al. A missense single-nucleotide polymorphism in a gene encoding a protein tyrosine phosphatase (PTPN22) is associated with rheumatoid arthritis. Am. J. Hum. Genet. 75, 330-337 (2004).
10. Kyogoku, C. et al. Genetic association of the R620W polymorphism of protein tyrosine phosphatase PTPN22 with human SLE. Am. J. Hum. Genet. 75, 504-507 (2004).
11. Hasegawa, K. et al. PEST Domain-Enriched Tyrosine Phosphatase (PEP) Regulation of Effector/Memory T Cells. Science (80-.). 303, 685-689 (2004).
12. Dai, X. et al. A disease-associated PTPN22 variant promotes systemic autoimmunity in murine models. J. Clin. Invest. 123, 2024-2036 (2013).
13. Denny, J. C. et al. Systematic comparison of phenome-wide association study of electronic medical record data and genome-wide association study data. Nat. Biotechnol. 31, 1102-1110 (2013).
14. Aran, D., Hu, Z. & Butte, A. J. xCell: Digitally portraying the tissue cellular heterogeneity landscape. Genome Biol. 18, (2017).
15. Newman, A. M. et al. Robust enumeration of cell subsets from tissue expression profiles. Nat. Methods 12, 453-457 (2015).
16. Bendall, S. C. et al. Single-cell mass cytometry of differential immune and drug responses across a human hematopoietic continuum. Science (80-.). 332, 687-696 (2011).
17. Van Gassen, S. et al. FlowSOM: Using self-organizing maps for visualization and interpretation of cytometry data. Cytom. Part A 87, 636-645 (2015).
18. Becht, E. et al. Dimensionality reduction for visualizing single-cell data using UMAP. Nat. Biotechnol. 37, 38-47 (2019).
19. Salvador, J. M. et al. Alternative p38 activation pathway mediated by T cell receptor-proximal tyrosine kinases. Nat. Immunol. 6, 390-395 (2005).
20. Salvador, J. M., Mittelstadt, P. R., Belova, G. I., Fornace, A. J. & Ashwell, J. D. The autoimmune suppressor Gadd45a inhibits the T cell alternative p38 activation pathway. Nat. Immunol. 6, 396-402 (2005).
21. Wherry, E. J. & Kurachi, M. Molecular and cellular insights into T cell exhaustion. Nature Reviews Immunology vol. 15 486-499 (2015).
22. Paley, M. A. et al. Progenitor and terminal subsets of CD8+ T cells cooperate to contain chronic viral infection. Science (80-.). 338, 1220-1225 (2012).
23. Blackburn, S. D., Shin, H., Freeman, G. J. & Wherry, E. J. Selective expansion of a subset of exhausted CD8 T cells by αPD-L1 blockade. Proc. Natl. Acad. Sci. U.S.A 105, 15016-15021 (2008).
24. Schickel, J. N. et al. PTPN22 inhibition resets defective human central B cell tolerance. Sci. Immunol. 1, (2016).
25. Vang, T. et al. LYP inhibits T-cell activation when dissociated from CSK. Nat. Chem. Biol. 8, 437-446 (2012).
26. Brownlie, R. J., Wright, D., Zamoyska, R. & Salmond, R. J. Deletion of PTPN22 improves effector and memory CD8+ T cell responses to tumors. JCI Insight 4, (2019).
27. Brownlie, R. J. et al. Resistance to TGFβ suppression and improved anti-tumor responses in CD8+ T cells lacking PTPN22. Nat. Commun. 8, (2017).
28. Chang, H.-H. et al. PTPN22 Modulates Macrophage Polarization and Susceptibility to Dextran Sulfate Sodium-Induced Colitis. J. Immunol. 191, 2134-2143 (2013).
29. Spalinger, M. R. et al. Loss of protein tyrosine phosphatase nonreceptor type 22 regulates interferon-γ-induced signaling in human monocytes. Gastroenterology 144, (2013).
30. Zhang, J. et al. The autoimmune diseaseg-associated PTPN22 variant promotes calpain-mediated Lyp/Pep degradation associated with lymphocyte and dendritic cell hyperresponsiveness. Nat. Genet. 43, 902-907 (2011).
31. Hoekstra, E., Peppelenbosch, M. P. & Fuhler, G. M. Meeting report europhosphatase 2015: Phosphatases as drug targets in cancer. Cancer Res. 76, 193-196 (2016).
32. Zhang, Z. Y. Drugging the undruggable: Therapeutic potential of targeting protein tyrosine phosphatases. Acc. Chem. Res. 50, 122-129 (2017).
33. Chen, Y. N. P. et al. Allosteric inhibition of SHP2 phosphatase inhibits cancers driven by receptor tyrosine kinases. Nature 535, 148-152 (2016).
34. Ellrott, K. et al. Scalable Open Science Approach for Mutation Calling of Tumor Exomes Using Multiple Genomic Pipelines. Cell Syst. 6, 271-281.e7 (2018).
35. Thorsson, V. et al. The Immune Landscape of Cancer. Immunity 48, 812-830.e14 (2018).
36. Charoentong, P. et al. Pan-cancer Immunogenomic Analyses Reveal Genotype-Immunophenotype Relationships and Predictors of Response to Checkpoint Blockade. Cell Rep. 18, 248-262 (2017).
37. Ho, W. J. et al. Multi-panel mass cytometry reveals anti-PD1 therapy-mediated B and T cell compartment remodeling in tumor-draining lymph nodes. JCI Insight (2019) doi:10.1172/jci.insight.132286.
38. Hartmann, F. J., Simonds, E. F. & Bendall, S. C. A Universal Live Cell Barcoding-Platform for Multiplexed Human Single Cell Analysis. Sci. Rep. 8, (2018).
39. Nowicka, M. et al. CyTOF workflow: differential discovery in high-throughput high-dimensional cytometry datasets. F1000Research 6, 748 (2019).
40. Enot, D. P., Vacchelli, E., Jacquelot, N., Zitvogel, L. & Kroemer, G. TumGrowth: An open-access web tool for the statistical analysis of tumor growth curves. Oncoimmunology 7, (2018).

1. Use of a PTPN22 inhibitor for inhibition of PTPN22 in one or more immune cells of a subject in need thereof.

2. Use of a PTPN22 inhibitor for treating cancer in a subject in need thereof.

3. The use of either claim 1 or 2, wherein the inhibitor of PTPN22 is selected from the group consisting of a PTPN22 antagonist, a PTPN22 gene suppressor, and a combination thereof.

4. The use of claim 3, wherein the inhibitor of PTPN22 is a PTPN22 antagonist.

5. The use of claim 1 or 2, wherein the inhibitor of PTPN22 is selected from the group consisting of: LTV1, a compound of Formula (I) 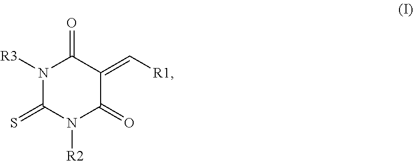
wherein R₁ is selected from the group consisting of 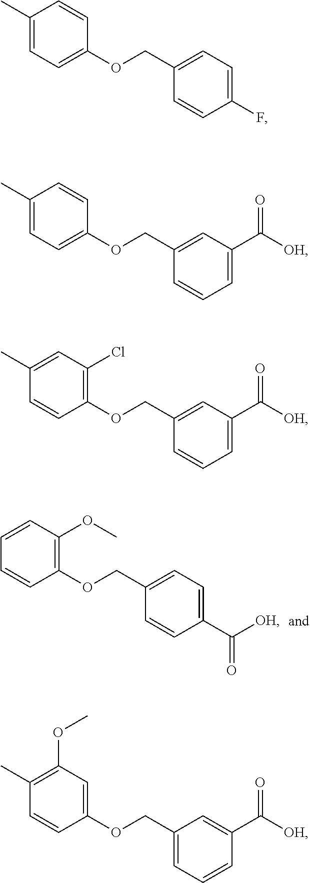
wherein R₂ and R₃ are the same or different and are each selected from the group consisting of H, CH₃, 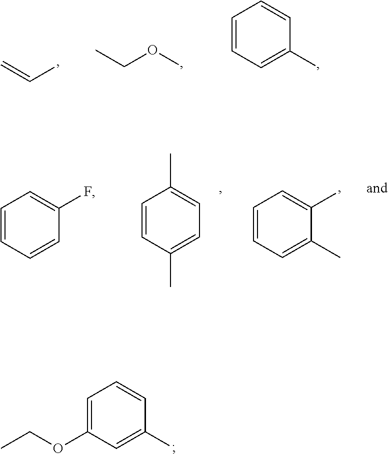
or a pharmaceutically acceptable salt, solvate, or stereoisomer thereof; [symbol]
a compound of Formula (II) 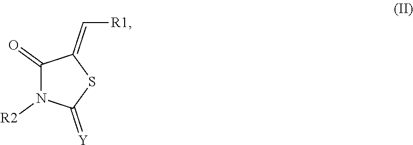
wherein R₁ is selected from the group consisting of H, CH₃, 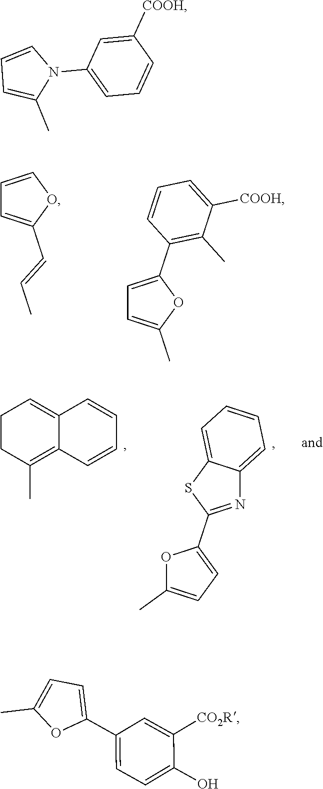
wherein R₂ is selected from the group consisting of H, CH₃, 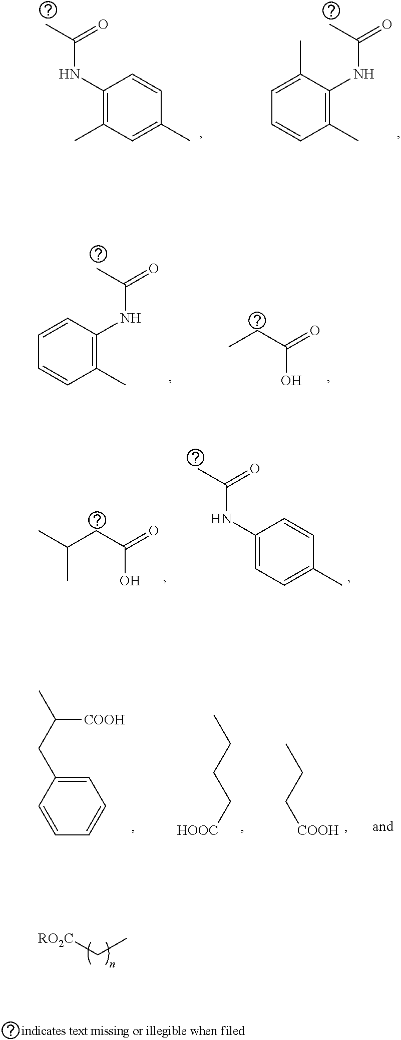
wherein N is 1 to 20, and Y is S or O, or a pharmaceutically acceptable salt, solvate, or stereoisomer thereof;
a compound of Formula (III)
R3P—Au—Cl,Au(I) (III) [symbol]
wherein R is selected from the group consisting of 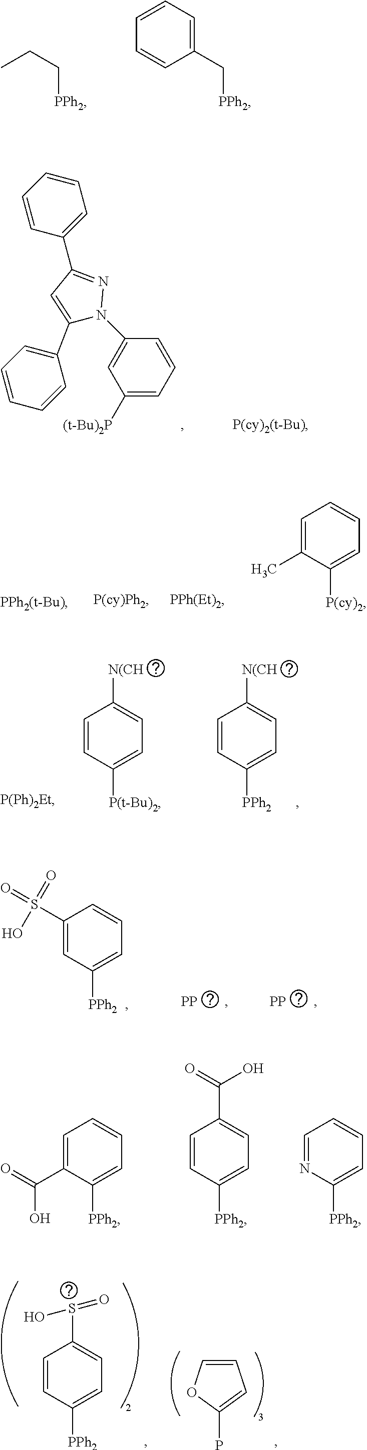 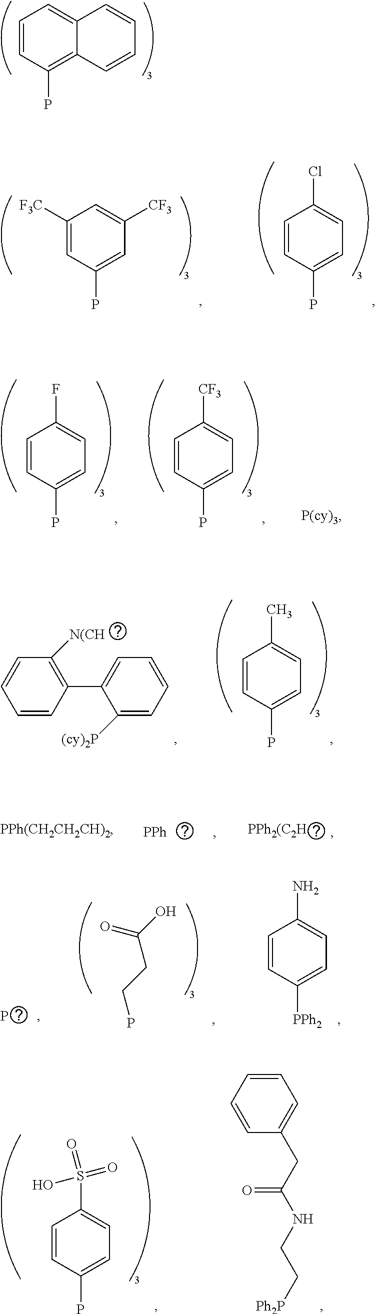 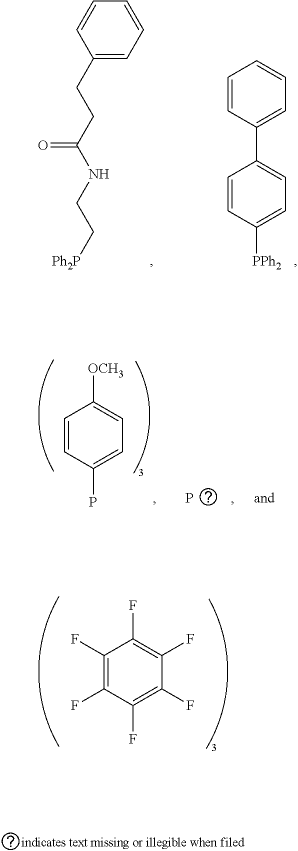
or a pharmaceutically acceptable salt, solvate, or stereoisomer thereof; and
a compound of Formula (IV), 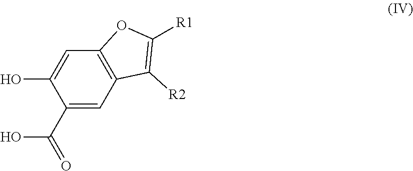
wherein R₁ is selected from the group consisting of 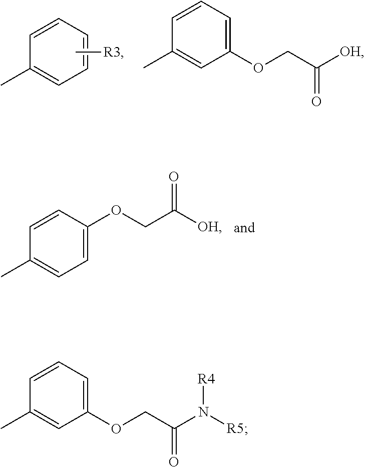
wherein R₂ is selected from the group consisting of 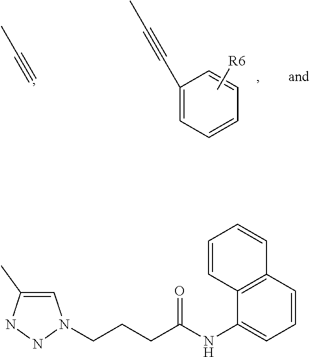
and wherein R₃=4-OCF₃, R₄= [symbol] , R₅= [symbol] , R₆=3-Cl; or a combination of these compounds thereof.

6. The use of claim 1 or 2, wherein the systemic inhibitor of PTPN22 is a PTPN22 gene suppressor.

7. The use of claim 6 wherein the PTPN22 gene suppressor is a PTPN22 siRNA.

8. The use of claim 2, wherein the cancer is a solid cancer.

9. The use of claim 8, wherein the solid cancer is selected from the group consisting of: anal, bladder, bone, breast, central nervous system, cervical, colon, endometrial, esophageal, gastric, head and neck, hepatobiliary, kidney, leukemia, lung, lymphoma, melanoma, merkel cell, ovarian, pancreatic, prostate, soft tissue sarcomas, testicular, thymoma, thyroid, or uterine cancer.

10. The use of claim 9, wherein the solid cancer is pancreatic cancer.

11. The use of claim 10, wherein the pancreatic cancer is metastatic.

12. The use of claim 1 or 2, wherein PTPN22 inhibitor is composed of a pharmaceutical composition comprising the systemic inhibitor of PTPN22 and a pharmaceutically acceptable carrier.

13. The use of claim 12, wherein the pharmaceutical composition further comprises a neoadjuvant, an adjuvant, or a combination thereof.

14. The use of claim 1 or 2, wherein PTPN22 inhibitor is composed of a pharmaceutical composition comprising the systemic inhibitor of PTPN22, at least one additional anti-cancer agent, and a pharmaceutically acceptable carrier.

15. The use of claim 15, wherein the anti-cancer agent is selected from the group consisting of a PD1 inhibitor, a PDL1 inhibitor; a CTLA4 inhibitor; a LAG3 inhibitor; IDO inhibitor; a co-stimulatory agonist therapy; a cancer vaccine; a T cell transfer therapy, and a combination thereof.

16. The use of claim 15, wherein the PD1 inhibitor and the PDL1 inhibitor is selected from the group consisting of pembrolizumab, nivolumab, durvalumab, atezolizumab, avelumab, and a combination thereof.

17. A compound of formula V: 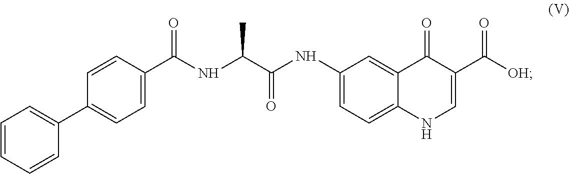 [SMILES: C[C@H](NC(=O)c1ccc(-c2ccccc2)cc1)C(=O)Nc1ccc2[nH]cc(C(=O)O)c(=O)c2c1]
or a salt, solvate, or stereoisomer thereof.

18. Use of a compound of formula V, or a salt, solvate, or stereoisomer thereof for inhibition of PTPN22 in one or more immune cells of a subject in need thereof.

19. Use of a compound of formula V, or a salt, solvate, or stereoisomer thereof for treatment of a cancer in a subject in need thereof.

20. The use of claim 18 or 19, wherein compound of formula V, or a salt, solvate, or stereoisomer thereof is in a pharmaceutical composition comprising a pharmaceutically acceptable carrier.

21. The use of claim 20, wherein the pharmaceutical composition further comprises, at least one additional anti-cancer agent.

22. The use of claim 21, wherein the at least one additional anti-cancer agent is selected from the group consisting of a PD1 inhibitor, a PDL1 inhibitor; a CTLA4 inhibitor; a LAG3 inhibitor; IDO inhibitor; a co-stimulatory agonist therapy; a cancer vaccine; a T cell transfer therapy, and a combination thereof.

23. The use of claim 22, wherein the PD1 inhibitor and the PDL1 inhibitor is selected from the group consisting of pembrolizumab, nivolumab, durvalumab, atezolizumab, avelumab, and a combination thereof.
